(19) United States
(12) Patent Application Publication
(10) Pub. No.: US 2004/0235027 A1
(43) Pub. Date: Nov. 25, 2004

(54) Preparation and application of ligand-biopolymer conjugates

(75) Inventors: Kit S. Lam, Davis, CA (US); Qingchai Xu, Dublin, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Regent of the University of California, Oakland, CA

(21) Appl. No.: 10/815,468

(22) Filed: Mar. 31, 2004

Related U.S. Application Data
(60) Provisional application No. 60/459,303, filed on Mar. 31, 2003.

(51) Int. Cl. C12Q 1/68; G01N 33/53; C12M 1/34

(52) U.S. Cl. 435/6; 435/7.1; 435/287.2

(57) ABSTRACT

The present invention provides methods for the site-specific attachment of ligands to a biopolymer. These methods are suitable for microarray construction, biodegradable scaffold formation, cell or tissue growth, as well as the conjugates or products formed by those methods.

### CROSSED-REFERENCE TO RELATED APPLICATION

This application claims the benefit of U.S. Provisional Patent Application Serial No. 60/459,303, filed Mar. 31, 2003, the content of which is incorporated herein by reference.

### STATEMENT AS TO RIGHTS TO INVENTIONS MADE UNDER FEDERALLY SPONSORED RESEARCH OR DEVELOPMENT

A portion of the present invention was made under federally sponsored research and development under National Institutes of Health Grant No. R33 CA 89706 and R33 CA86364. The Government may have rights in certain aspects of this invention.

### BACKGROUND OF THE INVENTION

Recent advances in such areas as tissue engineering, high throughput screening and general microarray technology has led to the investigation of new conjugates and materials that can be used in those research areas. For example, when small molecule ligands, peptides or peptide mimetics are attached to an appropriate inert matrix, the resultant conjugate can be used for a variety of applications depending on the matrix. A semi-solid matrix with appropriate ligands or proteins attached would find utility as a support for cell growth. Similarly, suitable ligands, peptides and the like, when attached to a suitable biodegradable scaffold or matrix could be used for tissue engineering.

Still other applications involve the use of microarrays having site-specifically attached small molecule ligands, candidate therapeutic agents, or peptides, useful for the development of diagnostics, therapeutics and tools for the analysis of the proteome (see Haab, B. B. et al. Genome Biol. 2001, 2:RESEARCH0004; Joos, T. O., et al. Electrophoresis 2000, 21:2641; Robinson, W. H. et al. Nat. Med. 2002, 8:295; Robinson, W. H. et al. Nat Biotechnol. 2003. 21:1033. Zhu, H., et al, Science 2001, 293:2101; Zhu, H. et al. Nat. Genet. 2000, 26:283).

Although considerable advances have been made on this subject (see Miller, J. C., et al. Proteomics 2003, 3:56, Pavlickova, P. et al. Biotechniques 2003, 34:124; Schweitzer, B. and Kingsmore, S. F. Curr. Opin. Biotechnol. 2002, 13:14), the use of protein microarrays in research and diagnostic settings are still limited. Several issues are important in developing peptide microrarrays. In most cases, the protein is immobilized on the slide via non-specific covalent binding (see Zhu, H. Nat. Genet. 2000, 26:283; Wilson, D. S. and Nock, S. Angew Chem. Int. Ed. Engl. 2003, 42:494; Miller, J. C., et al. Proteomics 2003, 3:56, Pavlickova, P. et al. Biotechniques 2003, 34:124; Schweitzer, B. and Kingsmore, S. F. Curr. Opin. Biotechnol. 2002, 13:14; MacBeath, G. and Schreiber, S. L. Science 2000, 289:1760). Site specific binding is required on a peptide microarray to immobilize the peptide with the correct orientation of the C— or N-terminus. Further, it is extremely difficult to control the amount of compound ligated directly to the solid support, which can vary from spot to spot, and from experiment to experiment.

What is needed in the art are new methods for the site-specific attachment of ligands to a biopolymer with high signal intensity, suitable for microarray construction or for biodegradable scaffold formation, as well as the conjugates or products formed by those methods. Surprisingly, the present invention provides such methods and conjugates.

### SUMMARY OF THE INVENTION

In one aspect, the present invention provides a microarray comprising a support having a plurality of discrete regions having a biopolymer spotted thereon, wherein attached to the biopolymer in each of the regions is a ligand that can be the same or different from a ligand in any other of the discrete regions, and wherein the concentration of the ligand in the discrete regions is substantially normalized.

In a second aspect, the present invention provides a method of producing a concentration-normalized ligand array, the method comprising: (a) forming a ligand-modified biopolymer by attaching a ligand to a functionalized biopolymer via chemoselective ligation; and (b) spotting an aliquot of the modified biopolymer mixture onto each of a plurality of discrete regions on a solid support to produce a concentration-normalized ligand array.

In a third aspect, the present invention provides a method for promoting cell or tissue growth at a desired site, the method comprising contacting the site with a ligand-modified biopolymer in an amount effective to promote cellular chemotaxis and cell or tissue growth at the site, wherein the biopolymer component is a member selected from the group consisting of agarose, polylysine and polyacrylamide, wherein the ligand component is a chemotactic peptide specific for a cell surface receptor, and wherein the ligand component is attached to the biopolymer component via chemoselective ligation.

In a fourth aspect, the present invention provides a method for assaying the binding of ligands to biological materials.

### BRIEF DESCRIPTION OF THE DRAWINGS

FIG. 1. Scheme showing the binding of a ligand to a biopolymer.

FIG. 2. Results from Jurkat cells binding assay corresponding to: A. No cells bound on gel surface of ketone-modified agarose (negative control); B. A few cells bound on gel surface of low peptide-loaded agarose (pLDIn-linked agarose, 0.02 mmol/g); C. A lot of cells bound on gel surface of 5% of higher peptide-loaded agarose (sppLDIn-linked agarose, 0.3 mmol/g) diluted in agarose. The final concentration of agarose gel is 1% in PBS.

FIG. 3. Results of micro adhesion assays of Jurkat cells on peptide microarray. A. Solutions of agarose conjugated to sppLDIn-Tdts-Dpr(Aoa)-NH₂ peptide with varying amount of peptide and agarose were printed on glass slide to form a microarray; B. 60 different peptide-agarose conjugate solutions were printed to form a microarray. All spots were made from a 1.5 mM peptide and 0.1 mg/mL modified agarose in 25% DMSO/acetate buffer, pH 4.5. Strong binding spots corresponding to: A1, A2, A3, D3, D10, E1, E3, E10, F2 and F3.

FIG. 4. Cell binding assay using Jurkat cells, and optimization of peptide ligation and microarray preparation: solutions of agarose conjugated to sppLDIn-Tdts-Dpr(Aoa)-NH₂ peptide with varying amount of peptide and agarose scaffold (ketone: 0.3 mmol/g) were printed on a glass slide to form a microarray.

FIG. 5. A: Chemical structure of the small molecule ligands comprising a peptidomimetic library to be printed on PVDF membrane as a microarray; B: Enzyme-linked colormetric binding assay of 300 μm microarrays stained with Streptavidin-alkaline phosphatase conjugate to detect streptavidin binding spots; and C: Enzyme-linked colormetric binding assay of 100 μm microarrays stained with Streptavidin-alkaline phosphatase conjugate to detect streptavidin binding spots.

FIG. 6. MALDI mass spectrometry analysis of protein samples corresponding to: A. Unmodified human serum album (HSA), average molecular weight 66804±100 Da; B. The ketone modified HSA (I, in Scheme 3), an average molecular weight 67337±200 Da, with an average loading of 5.4 ketones/protein; C. Peptide-HSA conjugate (peptide: sppLDIn-Tdts-Dpr(Aoa)-NH₂), an average molecular weight 73173±400 Da, with an average loading of 5.2 peptides/molecule of HSA.

FIG. 7. Polyacrylamide gel electrophoresis verifies conjugation of peptides to HSA. HSA and peptide-HSA conjugate (sppLDIn-HSA) were subjected to; A. 10% SDS PAGE separation of conjugate and unmodified HSA with colloidal Coomassie blue staining; B. 2D PAGE analysis of HSA and peptide-HSA conjugate with Coomassie blue staining of gels. Separate gels were run for each sample. Scans of gels are overlaid, with alignment of molecular weight markers, so that direct comparison between the HSA and peptide-HSA conjugate can be made.

FIG. 8. Results from Jurkat cells binding assay of an array of 60-aminooxy peptides conjugated on the modified HSA. All spots were made from a 10 82 M peptide and 0.1 mg/mL modified HSA in 25% DMSO/acetate buffer, pH 4.5. Strong binding spots corresponding to: A1, D10, E1, E3, E9, E10, F2, F3 and F10.

FIG. 9. Results from cell binding & biotin-detection assay from two duplicated slides corresponding to; A. slide subjected to avidin-horseradish peroxidase (Hrp) detection; B. slide subjected to Jurkat cell binding assay; C. a cell-bound spot (high magnification) taken from slide B. R1: 8 spots were made from biotin-HSA conjugate (0.5 mg/mL); R2: 8 spots were made from sppLDIn-HSA conjugate (0.5 mg/mL).

FIG. 10. Results of micro adhesion assays of Jurkat cells on peptide microarray. Solutions of HSA conjugated to sppLDIn-Tdts-Dpr(Aoa)-NH₂ peptide with varying amount of peptide and HSA were printed on plastic slide to form a microarray. Spots H₁-H₁₂ are ˜0.1-0.2 μg/mL poly-lysine in PBS buffer.

FIG. 11. Synthesis of peptide agarose conjugate on microarray. Peptide synthesis was performed on resin. Dpr(Boc-Aoa) and a hydrophilic spacer was incorporated between resin and peptide. The hydroxyl group on agarose reacts with levulinic acid to form an ester; ketones on modified agarose bind to the amino-oxy groups of Aoa and form oximes. After this conjugation to agarose, xenobiotics (R) were added to the lysine of the peptide.

FIG. 12. Detection of antibody against lipoic acid and xenobiotics. Twenty three xenobiotics and lipoic acid coupled to either the 12 mer, PDC peptide, mutant PDC peptide and/or control albumin peptide were spotted. Reactivity was determined using A. mAb against PDCE2 (2H4) and B. mAb murine IgG control.

FIG. 13. Comparison between ELISA and microarray assay. Rabbit sera (n=5) at one month post-immunization were serially diluted (1:250, 1:750, 1:2250, 1:6750 and 1:20250) and IgG reactivity to small morecule-peptide-agarose conjugates were determined by both ELISA (B) and the microarray assay (A).

### DETAILED DESCRIPTION OF THE INVENTION

### I. ABBREVIATIONS AND DEFINITIONS

The following abbreviations are used in the present invention:

AMA: antimitochondrial antibodies;

Aoa: amino-oxyacetic acid;

Boc: tert-butoxycarbonyl;

BODIPY: 4,4-difluoro-5,7-dimethyl-4-bora-3_{α},4_{α}-diaza-s-indacene propionic acid;

BSA: bovine serum albumin;

DAPI: 4′,6-diamidino-2-phenylindole;

DCC: dicyclohexylcarbodiimide;

DCM: dichloromethane;

ddH₂O: double distilled water;

DIC: diisopropylcarbodiimide;

DIEA: N,N-diisopropylethylamine;

DMAP: 4-dimethylaminopyridine;

DME: 1,2-dimethoxyethane;

DMF: N,N-dimethylformamide;

DMSO: dimethylsulfoxide;

Dpr: diaminopropionic acid;

Dpr(Aoa): N-β-(amino-oxyacetyl)-L-diaminopropionic acid;

ELISA: enzyme-linked immunosorbent assay;

ESMS: electrospray ionization mass spectrometry;

Fmoc: 9-fluorenylmethoxylcarbonyl;

Fmoc-Dpr(Boc-Aoa): N^{α}-Fmoc-(N^{β}-Boc-amino-oxyacetyl)-L-diaminopropionic acid;

HOAc: acetic acid;

HOBt: N-hydroxybenzotriazole;

HOSu: N-hydroxysuccinimide;

HPLC: high performance liquid chromatography;

HRP: horseradish peroxidase;

HSA: human serum albumin;

IgG: immunoglobulin G;

KLH: keyhole limpet hemocyanin;

MALDI: matrix assisted laser desorption/ionization (mass spectrometry);

NHS: N-hydroxysuccinimide;

NMR: nuclear magnetic resonance;

OBOC: one-bead one compound combinatorial approach;

PBC: primary biliary cirrhosis;

PBS: phosphate buffered saline;

PBST: phosphate buffer saline with Tween 20;

PDC: pyruvate dehydrogenase complex;

PDVF: polyvinylidenedifluoride;

SDS PAGE: sodium dodecyl sulfate polyacrylamide gel electrophoresis;

Tdts: 4,7,10-trioxa-1,13-tridecanediamine succinimic acid;

TFA: trifluoroacetic acid;

TEMPO: 2,2,6,6-tetramethylpipelidine-1-oxyl radical;

TIS: triisopropylsilane;

TLC: thin-layer chromatography; and

2D PAGE: two dimensional polyacrylamide gel electrophoresis.

The natural amino acids used in the present invention are referred to herein by their common single letter abbreviations, wherein a capital letter refers to the L isomer, and a lower case letter refers to the D isomer.

The term “biopolymer” is defined as either a naturally occurring polymer, or a synthetic polymer that is compatible with a biological system or that mimics naturally occurring polymers. For example, and not by way of limitation, biopolymers of the present invention include oligosaccharides, proteins, polyketides, peptoids, hydrogels, poly(glycols) such as poly(ethylene glycol), and polylactates.

The terms “array” and “microarray” are used interchangeably, and are each intended to include a solid support having a suitable ligand immobilized on at least one spatially distinct region of its surface. An array can contain any number of ligands immobilized within any number of spatially distinct regions. The spacing and orientation of the ligands can be regular, e.g., in a rectangular or hexagonal grid, or the pattern can be irregular or random. In a particular embodiment, non-identical ligands are arranged in a regular pattern on the surface of a solid support and are useful, for example, in binding assays to determine whether analytes (capable of binding to selected ligands) are present in a sample. Ligands capable of detecting the presence of a component can be placed in a spatially distinct region, so that in a single analysis, a determination can be made as to whether one or more of the components of the set are contained within the sample.

The terms “sample” or “target analyte” are meant to include component mixtures which can contain the target molecule. The test sample can be obtained from a biological source (e.g., a physiological fluid, including, blood, saliva, ocular lens fluid, cerebral spinal fluid, sweat, urine, peritoneal fluid, amniotic fluid, and the like) or can be the product of fermentation broths, cell cultures, cell and tissue extract, chemical reaction mixtures, and the like. Additionally, the sample can be used directly as obtained or following pretreatment, such as preparing plasma from blood, diluting viscous fluids, and the like. Other methods of treatment can involve filtration, distillation, extraction, concentration, inactivation of interfering components, and the addition of reagents. In addition, a solid material, such as cells, which can contain the target molecule, can be used as the sample. In some instances, it may be beneficial to modify a solid test sample to form a liquid medium or to release a target molecule (e.g., via lysing of cells). In still other cases, the sample can be a viral peptide, bacteria, yeast, parasites or intact cells.

The term “chemoselective ligation” refers to the controlled and predetermined attachment of a first component to a second component, due to specifically matched functional groups in the first and second components. Specifically matched functional groups are those functional groups that can react with each other to form a covalent linkage, but will be relatively unreactive with other functional groups present in either the first or second component.

The terms “support” and “solid support” refer to a member that is a solid, typically insoluble, medium to which the biopolymer of the present invention is attached. Supports useful in the present invention include, for example, glass, polystyrene, PDVF membranes, nylon membranes, and polycarbonate slides.

The term “ligand” refers to a molecule that selectively binds, covalently or noncovalently, to another specific molecule or to a specific part of a molecule.

The term “normalized” refers to a state wherein each discrete region has the same concentration of sample as all the other regions.

The term “noncovalent interactions” refers to the interaction of two species in close proximity that does not form a covalent bond. Types of noncovalent interactions include, for example, hydrogen bonding, van der Waals interaction, coordination, pi-pi interaction, hydrophobic interactions and hydrophilic interactions.

The term “covalent interaction” refers to the interaction of two species in close proximity that form a covalent bond.

The term “aliquot” refers to a measured subset of the whole sample.

The term “chemotaxis” refers to the orientation or movement of an organism or cell either towards or away from a particular site, in relation to chemical agents. The term “chemotactic” refers to an agent that has the property of chemotaxis.

### II. GENERAL

The use of site-specific chemoselective ligation for the attachment of ligands to a biopolymer provides conjugates having a wide variety of utilities. For example, suitable attachment of cell adhesion peptides to agarose can now be accomplished to provide a temperature sensitive reversible gel matrix that can support cell attachment and growth. Alternatively, peptides can be attached to a biopolymer, such as agarose, in a site-specific manner and then spotted on a solid support to form a microarray for such purposes as cell-binding assays and protein-binding assays.

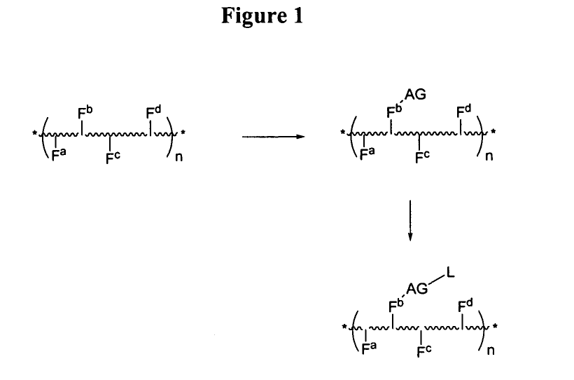

The general process for chemoselective ligation is illustrated in FIG. 1. In this figure, the wavy line represents a biopolymer having “n” subunits that can be the same or different. The subunits will generally have one or more functional groups (shown as F^{a}, F^{b}, F^{c}, F^{d}, etc.) that can be reacted with suitable attaching groups (AG) independently. In FIG. 1, the functional group F^{b} is shown to be conjugated to AG, while the remaining functional groups are unreacted. The attaching group contains functionality that is selected for a specific functional group on the biopolymer and can therefore react in a controlled and predetermined manner. Additionally, the attaching group (AG) has, or is subsequently modified to have, a functional group that can be reacted specifically with a suitable ligand (L) such as, for example, a peptide, small molecule, diagnostic agent, a pharmaceutical agent or candidate. The use of such site specific modification with attaching groups and ligands provides conjugates having a predetermined concentration of ligand. The resulting ligand-biopolymer conjugates can then be used, in one group of embodiments, to prepare microarrays having normalized concentrations of various ligands.

### III. MICROARRAYS AND METHODS FOR SPOTTING BIOPOLYMERS

A. Microarrays

In view of the above, the present invention provides in one aspect, a microarray comprising a support having a plurality of discrete regions having a biopolymer spotted thereon, wherein attached to the biopolymer in each of the regions is a ligand that can be the same or different from a ligand in any other of the discrete regions, and wherein the concentration of the ligand in the discrete regions is substantially normalized.

The supports utilized in preparing the microarrays of the present invention can be prepared from a variety of materials including, for example, glass, polystyrene, PDVF membranes, nylon membranes and polycarbonate slides. Other suitable plastic materials include crystalline thermoplastics (e.g., high and low density polyethylenes, polypropylenes, acetal resins, nylons and thermoplastic polyesters) and amorphous thermoplastics (e.g., polycarbonates and poly(methyl methacrylates). Selection of suitable plastic or glass materials will generally depend on the ultimate use of the microarray and consider the combination of such properties as rigidity, toughness, resistance to long term deformation and resistance to thermal degradation. One of skill in the art will appreciate that other supports are useful in the present invention.

The solid support utilized in the present invention will also have a plurality of discrete regions. These can be in the form of, for example, wells (e.g. 96-, 388- or 1552-well plates), or planar regions on a slide. These regions (e.g., discrete spots) on the slide will generally be circular in shape, with a typical diameter of between about 10 microns and about 500 microns (and preferably between about 20 and about 200 microns). The regions are also preferably separated from other regions in the array by about the same distance (e.g., center to center spacing of about 20 microns to about 1000 microns).

Biopolymers useful in the present invention are characterized by having a functional group that can undergo chemoselective ligation with a complementary functional group in the presence of a plurality of similar functional groups. For example, and not by way of limitation, a primary alcohol, as shown in Schemes 1 and 2, can be selectively reacted in the presence of secondary alcohols. In a similar manner, a polyamine biopolymer could have both primary and secondary amines, wherein only the primary amines undergo chemoselective ligation with an appropriate complementary functional group. Other similar functional groups with a preference in reactivity for one over the other could be aldehydes and ketones. In some cases, the biopolymer may not itself comprise a functional group for chemoselective ligation, but may be subsequently derivatized with a functional group for chemoselective ligation.

A variety of biopolymers are useful in the present invention. Particularly useful are biopolymers such as, for example, oligosaccharides (e.g., agarose), proteins (e.g., human serum albumin), polyketides, peptoids, hydrogels, polylactates and polyurethanes. One of skill in the art will appreciate that other biopolymers are useful in the present invention.

Depending on the solid support and the properties of the desired array, the biopolymer will generally be attached to the support via noncovalent interactions. Non-covalent attachment can be accomplished by, for example, spotting the biopolymer onto the support with attachment to the functional groups of the support occurring through hydrogen bonding, via van der Waals interactions, hydrophobic interactions, hydrophilic interactions and combinations thereof. In other embodiments, the biopolymer will be attached to the support structure via covalent interactions.

Ligands that are useful in the present invention include, for example, amino acids, peptides, proteins, sugars, lipids, nucleic acids, small organic compounds, pharmaceutical agents, candidate pharmaceutical agents, natural or synthetic antigens, and combinations thereof.

Amino acids useful in the present invention include both natural and non-natural amino acids. The amino acids of the present invention can be further derivatized with, for example, protecting groups known to one of skill in the art. Several amino acids can also be linked together in a chain to form a peptide. Peptides useful in the present invention can have between 2 and 5 amino acids. Other peptides useful in the present invention can have between 6 and 20 amino acids. Even further peptides useful in the present invention can have between 21 and 50 amino acids. Furthermore, several peptides can be linked together to form a protein. Proteins useful in the present invention can have between 2 and 5 peptides. In other aspects, the proteins of the present invention can have between 6 and 10 peptides. In still other aspects, the proteins of the present invention can have between 11 and 20 peptides. In yet another aspect, the proteins of the present invention can have between 21 and 100 peptides. One of skill in the art will appreciate that other peptides and proteins are useful in the present invention.

Sugars useful as ligands in the present invention include, for example, glucose, ribose, galactose and fructose. These sugars can be cyclic or non-cyclic, of which the cyclic form can be the α- or β-anomer; or the sugars can be derivatized via reductive methods, via formation of a hemiacetal or acetal, by formation of an acetate group, or by replacing an alcohol with an amine. The sugars can be further derivatized through the removal of a hydroxy group, to form the deoxy-sugar. The sugars of the present invention can also be linked together to form oligosaccharides such as sucrose, maltose, cellulose, starch and glycogen. One of skill in the art will appreciate that further derivatization of sugars can be carried out.

Ligands of the present invention can also comprise nucleic acids. Nucleic acids are polymers comprised of many individual components, nucleotides, linked together. Each nucleotide is composed of a phosphate, a sugar and an amine base. The sugars can be those discussed previously. Preferred sugars useful for nucleic acids include ribose and deoxy-ribose. Amine bases useful for nucleic acids include, for example, purines such as adenine and guanine, as well as pyrimidines such as cytosine, uracil and thymine. Other sugars and bases useful in nucleic acids of the present invention will be known to one of skill in the art. In a preferred aspect, the nucleic acids useful in the present invention are paired with a complementary nucleic acid in a double helix conformation.

In another preferred aspect, the ligands of the present invention are an antigen to which antibodies from the serum of a patient will bind. These antigen microarrays can be used as diagnostics. Antigens useful in the present invention include, but are not limited to, peptides, sugars, glycopeptides, lipids, glycolipids, and proteins.

Lipids useful in the present invention include, for example, fats, waxes and steroids. These lipids are characterized as being soluble in organic solvents, such as hexanes, and not water. Preferred fats of the present invention comprise a tri-ester with carbon chains of between 5 and 25 carbons each. Preferred waxes of the present invention comprise a single ester with carbon chains of between 10 and 50 carbons each. Preferred steroids of the present invention include cholesterol, for example. In some aspects, lipids of the present invention can additionally comprise a phosphate group. One of skill in the art will appreciate that other lipids are useful in the present invention.

Small organic molecules useful in the present invention are comprised of, for example, carbon, hydrogen, oxygen, nitrogen and sulfur. In some aspects, the small organic molecules may additionally comprise silicon, phosphorous, boron and a halogen, for example. Preferred small organic molecules have a molecular weight of less than 750. More preferred small organic molecules have a molecular weight of less than 500. Even more preferred small organic molecules have a molecular weight of between 200 and 400. One of skill in the art will appreciate that further elements can be incorporated in the small organic molecules.

Pharmaceutical agents according to the invention include agents that affect any biological process. The term “drug” or “therapeutic agent” refers to an active agent that has a pharmacological activity or benefits health when administered in a therapeutically effective amount. Examples of drugs or therapeutic agents include substances that are used in the prevention, diagnosis, alleviation, treatment or cure of a disease or condition. Candidate pharmaceutical agents include drugs and drug conjugates that are useful for the treatment of a disease state or condition, but are still in a developmental stage. One of skill in the art will appreciate that further ligands are useful in the present invention.

Additionally, the biopolymers have one or more attached ligands wherein each ligand is attached to the biopolymer via chemoselective ligation. By utilizing chemoselective ligation, functional groups can be introduced into the biopolymer in a predetermined amount, for example, by reaction with known functional groups present in the biopolymer. Specific examples of introducing functional groups into a biopolymer are described below for agarose and for human serum albumin. One of skill in the art will appreciate that a number of other methods could be similarly employed. The requirements for chemoselective ligation are that the biopolymer possesses at least one functional group that can be reacted, generally in the presence of other functional groups.

Typically, the chemoselective ligation functional groups are an electrophile-nucleophile pair, although other pairings will be apparent to one of skill in the art. In an electrophile-nucleophile pair, the electrophile can be, for example, a ketone, an aldehyde, or an α-halo carbonyl. In these pairings, the nucleophile can be, for example, an amine, a thiol, an alcohol, a hydrazide, an aminooxy group, a thiosemicarbazide, a β-amino thiol, a carboxylate, or a thiocarboxylate. In one aspect, the biopolymer can comprise the nucleophile, and the ligand can comprise the electrophile. In other aspects, the biopolymer can comprise the electrophile, and the ligand can comprise the nucleophile.

Preferred pairings of electrophile and nucleophile useful in the present invention are shown below (Lemieux, G. A. et al. Trends in Biotechnology 1998, 16, 506; Shin, I. et al. Bull. Korean Chem. Soc. 2000, 21(9), 845).

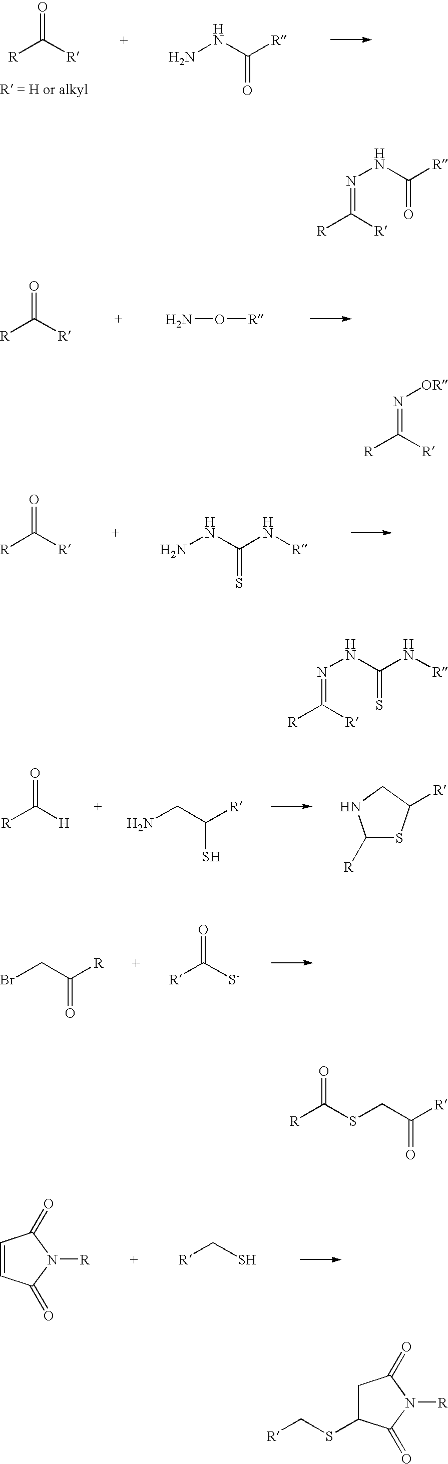

One of skill in the art will appreciate that other nucleophiles, such as alcohols, phosphorous based nucleophiles and carbon-based nucleophiles are useful in the present invention. In addition, one of skill in the art will appreciate that other electrophiles, such as α,β-unsaturated ketones, anhydrides and esters, for example, are useful in the present invention.

In a preferred aspect, the present invention provides a microarray wherein the biopolymer is agarose and the support is glass. In another preferred aspect, the biopolymer is human serum albumin, and the support is polystyrene.

In another preferred aspect, the present invention provides a microarray where the difference in concentration between any two discrete regions is less than 50%. In a more preferred aspect, the present invention provides a microarray where the difference in concentration between any two discrete regions is less than 20%. In a most preferred aspect, the present invention provides a microarray where the difference in concentration between any two discrete regions is less than 5%.

B. Methods of Preparing Microarrays

In a related aspect, the present invention provides methods of producing a concentration-normalized ligand array, the method comprising: (a) forming a ligand-modified biopolymer by attaching a ligand to a functionalized biopolymer via chemoselective ligation; and (b) spotting the ligand-modified biopolymer onto each of a plurality of discrete regions on a solid support in sufficient amounts to produce a concentration-normalized ligand array.

In optional, but preferred embodiments, the invention further comprises, prior to step (b), step (a)(i) combining the ligand-modified biopolymer with a biopolymer solution to form a modified biopolymer mixture.

Solid supports that are useful in the present invention include, for example, glass, polystyrene, PDVF membranes, nylon membranes, and polycarbonate slides. One of skill in the art will appreciate that further supports are useful in the present invention.

In a preferred aspect, the aliquot is spotted onto the solid support under conditions sufficient to form a gel-coated surface.

Biopolymers of the present invention are selected from the group consisting of oligosaccharides, proteins, polyketides, peptoids, hydrogels, polylactates and polyurethanes. One of skill in the art will appreciate that further biopolymers are useful in the present invention.

The ligands of the present invention are selected from the group consisting of amino acids, peptides, proteins, sugars, lipids, nucleic acids, glycopeptides, glycolipids, small organic compounds, pharmaceutical agents, candidate pharmaceutical agents and combinations thereof. One of skill in the art will appreciate that further ligands are useful in the present invention.

In a preferred aspect, the present invention provides a method wherein the ligand-modified biopolymer is peptide-modified agarose and the solid support is glass.

In another preferred aspect, the present invention provides a method wherein the ligand modified biopolymer is peptide-modified human serum albumin and the solid support is polystyrene.

The preferred microarrays of the present invention are prepared using agarose (low melting) which can be chemically modified with a ketone (Schemes 1 and 2). A synthetic peptide containing an aminooxy group can then be conjugated onto the modified agarose at the ketone moiety via oxime chemoselective ligation reaction. In this reaction, only the aminooxy group, but not the other free amines or sulfhydryl groups in the peptide, reacts with the ketone group in the agarose. The peptide-linked agarose solution melts above 60° C. but gels at 25° C. Depending on the composition and type of agarose that is utilized, the melting and gelling temperature can vary. If diluted, the agarose will not gel, but rather will dry and stick on the substrate surface. Following ligation, the peptide-agarose solutions can then be spotted onto a substrate with an automatic arrayer. After overnight drying, the peptide microarray is ready for biological studies.

C. Spotting of Functionalized Biopolymers

A variety of methods can be utilized for spotting functionalized biopolymers onto a solid support, including mechanical microspotting, ink jet techniques and in some instances, photolithography. Each of these methods can be automated and applied to microarray production.

Microspotting encompasses deposition technologies that enable automated microarray production by printing small quantities of pre-made biochemical substances onto solid surfaces. Printing is accomplished by direct surface contact between the printing substrate and a delivery mechanism, such as a pin or a capillary. Robotic control systems and multiplexed printheads allow automated microarray fabrication.

Ink jet technologies utilize piezoelectric and other forms of propulsion to transfer biochemical substances from miniature nozzles to solid surfaces. Using piezoelectricity, the sample is expelled by passing an electric current through a piezoelectric crystal which expands to expel the sample. Piezoelectric propulsion technologies include continuous and drop-on-demand devices. In addition to piezoelectric ink jets, heat may be used to form and propel drops of fluid using bubble-jet or thermal ink jet heads, however, such thermal ink jets are typically not suitable for the transfer of biological materials due to the heat which is often stressful on biological samples. Examples of the use of ink jet technology include U.S. Pat. No. 5,658,802.

With photolithography, a glass wafer, modified with photolabile protecting groups is selectively activated and a suitable biopolymer can then be synthesized on the arrays, or brought into contact with an activated surface.

D. Microarray Analysis

The methods of screening the microarrays of the present invention identify ligands within the microarray that demonstrate a biological activity of interest, such as binding, stimulation, inhibition, toxicity, taste, etc. Other microarrays can be screened according to the methods described infra for enzyme activity, enzyme inhibitory activity, and chemical and physical properties of interest. Many screening assays are well known in the art; numerous screening assays are also described in U.S. Pat. No. 5,650,489.

The ligands discovered during an initial screening may not be the optimal ligands. In fact, it is often preferable to prepare a second microarray based on the structures of the ligands selected during the first screening. In this way, one may be able to identify ligands of higher activity.

Binding Assays

The present invention allows identification of ligands that bind to acceptor molecules. As used herein, the term “acceptor molecule” refers to any molecule which binds to a ligand. Acceptor molecules can be biological macromolecules such as antibodies, receptors, enzymes, nucleic acids, or smaller molecules such as certain carbohydrates, lipids, organic compounds serving as drugs, metals, etc.

The ligands in microarrays of the present invention can potentially interact with many different acceptor molecules. Since the ligands are spatially addressable, the chemical identity of the ligands for a specific acceptor molecule can be determined.

If different color or identification schemes are used for different acceptor molecules (e.g., with fluorescent reporting groups such as fluorescein (green), Texas Red (Red), DAPI (blue) and BODIPY tagged on the acceptors), and with suitable excitation filters in the fluorescence microscope or the fluorescence detector, different acceptors (receptors) can be evaluated simultaneously to facilitate rapid screening for specific targets. These strategies not only reduce cost, but also increase the number of acceptor molecules that can be screened.

In the method of the present invention, an acceptor molecule of interest is introduced to the microarray where it will recognize and bind to one or more ligand species within the microarray. Each ligand species to which the acceptor molecule binds can be readily identified.

In addition to using soluble acceptor molecules, in another embodiment, it is possible to detect ligands that bind to cell surface receptors using intact cells. The use of intact cells is preferred for use with receptors that are multi-subunit or labile or with receptors that require the lipid domain of the cell membrane to be functional. The cells used in this technique can be either live or fixed cells. The cells can be incubated with the microarray and can bind to certain peptides in the microarray to form a “rosette” between the target cells and the relevant ligand spot.

Alternatively, one can screen the microarray using a panning procedure with cell lines such as (i) a “parental” cell line where the receptor of interest is absent on its cell surface; and (ii) a receptor-positive cell line, e.g., a cell line which is derived by transfecting the parental line with the gene coding for the receptor of interest. Differential binding of cells to a specific ligand spot on two or more microarray sets will enable one of skill in the art to identify the ligand specific to the receptor of interest.

As an alternative to whole cell assays for membrane bound receptors or receptors that require the lipid domain of the cell membrane to be functional, the receptor molecules can be reconstituted into liposomes where reporting group or enzyme can be attached.

In one embodiment, the acceptor molecule can be directly labeled. In another embodiment, a labeled secondary reagent can be used to detect binding of an acceptor molecule to a ligand of interest. Binding can be detected by in situ formation of a chromophore by an enzyme label. Suitable enzymes include, but are not limited to, alkaline phosphatase and horseradish peroxidase. In a further embodiment, a two color assay, using two chromogenic substrates with two enzyme labels on different acceptor molecules of interest, can be used. Cross-reactive and singly-reactive ligands can be identified with a two-color assay.

In specific examples, enzyme-chromogen labels and fluorescent (e.g. fluorescein isothiocyanate, FITC) labels are used.

In another embodiment, the ligand(s) with the greatest binding affinity can be identified by progressively diluting the acceptor molecule of interest until binding to only a few solid phase support beads of the microarray is detected. Alternatively, stringency of the binding with the acceptor molecule, can be increased. One of ordinary skill would understand that stringency of binding can be increased by (i) increasing solution ionic strength; (ii) increasing the concentration of denaturing compounds such as urea; (iii) increasing or decreasing assay solution pH; (iv) use of a monovalent acceptor molecule; (v) inclusion of a defined concentration of known competitor into the reaction mixture; and (vi) lowering the acceptor concentration. Other means of changing solution components to change binding interactions are well known in the art.

In another embodiment, ligands that demonstrate low affinity binding may be of interest. These can be selected by first removing all high affinity ligands and then detecting binding under low stringency or less dilute conditions.

Bioactivity Assays

The instant invention further provides assays for biological activity of a ligand-candidate from a microarray. The biological activities that can be assayed include toxicity and killing, stimulation and growth promotion, signal transduction, biochemical and biophysical changes, and physiological change.

It will further be understood by one of ordinary skill in the art that any cell that can be maintained in tissue culture, either for a short or long term, can be used in a biological assay. The term “cell” as used here is intended to include prokaryotic (e.g., bacterial) and eukaryotic cells, yeast, mold, and fungi. Primary cells or lines maintained in culture can be used. Furthermore, applicants envision that biological assays on viruses can be performed by infecting or transforming cells with virus. For example, and not by way of limitation, the ability of a ligand to inhibit lysogenic activity of lambda bacteriophage can be assayed by identifying transfected E. coli colonies that do not form clear plaques when infected.

Methods of the present invention for assaying activity of a ligands molecule of a microarray are not limited to the foregoing examples; any assay system that can be modified to incorporate the presently disclosed invention are useful.

Enzyme Mimics/Enzyme Inhibitors

The present invention further comprises microarrays that are capable of catalyzing reactions, i.e., enzyme microarrays; microarrays of molecules that serve as co-enzymes; and microarrays of molecules that can inhibit enzyme reactions. Thus, the present invention also provides methods to be used to assay for enzyme or co-enzyme activity, or for inhibition of enzyme activity.

Enzyme activity can be observed by formation of a detectable reaction product. In a particular embodiment, an enzyme from an enzyme microarray catalyzes the reaction catalyzed by alkaline phosphatase, e.g., hydrolysis of 5-bromo-4-chloro-3-indoyl phosphate (BCIP) and forms a blue, insoluble reaction product.

Co-enzyme activity can be observed by assaying for the enzyme activity mediated by a co-enzyme, where the natural or common co-enzyme is absent.

It is well known to one of ordinary skill in the art that a ligand that demonstrates enzyme activity, co-enzyme activity, or that inhibits enzyme activity, can be a peptide, a peptide mimetic, or one of a variety of small-molecule compounds.

### IV. CELLULAR CHEMOTAXIS AND CELL OR TISSUE GROWTH ON A LIGAND-MODIFIED BIOPOLYMER

In another aspect, the present invention provides a method for promoting cell or tissue growth at a desired site, the method comprising contacting the site with a ligand-modified biopolymer in an amount effective to promote cellular chemotaxis and cell or tissue growth at the site, wherein the biopolymer component is a member selected from the group consisting of agarose, polylysine and polyacrylamide, wherein the ligand component is a chemotactic peptide specific for a cell surface receptor, and wherein the ligand component is attached to the biopolymer component via chemoselective ligation.

A variety of biopolymers are useful in the present invention. Particularly useful are biopolymers such as, for example, oligosaccharides (e.g., agarose), proteins (e.g., human serum albumin), polyketides, peptoids, hydrogels, polylactates and polyurethanes. One of skill in the art will appreciate that other biopolymers are useful in the present invention. In a preferred aspect of the present invention, the biopolymer is agarose.

Additionally, the biopolymers have one or more attached ligands wherein each ligand is attached to the biopolymer via chemoselective ligation. By utilizing chemoselective ligation, functional groups can be introduced into the biopolymer in a predetermined amount, for example, by reaction with known functional groups present in the biopolymer. Specific examples of introducing functional groups into a biopolymer are described below for agarose and for human serum albumin. One of skill in the art will appreciate that a number of other methods could be similarly employed. The requirements for chemoselective ligation are that the biopolymer possesses at least one functional group that can be reacted, generally in the presence of other functional groups.

Ligands that are useful in the present invention include, for example, amino acids, peptides, proteins, sugars, lipids, nucleic acids, small organic compounds, pharmaceutical agents, candidate pharmaceutical agents, natural or synthetic antigens, and combinations thereof.

A matrix with an appropriate ligand can stimulate and support cell growth by providing a three-dimensional adherence environment. This three-dimensional matrix is also useful for supporting cell growth and for producing biomedically useful factors. In addition, the matrix provides a unique growth environment for unique cells, such as stem cells. One of skill in the art will appreciate that other cells are useful in the present invention.

Tissues that can be prepared by the methods of the present invention include, for example, skin, muscle, bone, nervous system, and organ tissue. One of skill in the art will appreciate that other tissues are useful in the present invention.

In another preferred aspect, the site is a member selected from the group consisting of a stent, a graft, an organ, a tissue and an implant. One of skill in the art will appreciate that other sites are useful in the present invention. In a further preferred embodiment, the cell or tissue growth occurs in vivo. In yet another preferred aspect, the cell or tissue growth occurs in vitro.

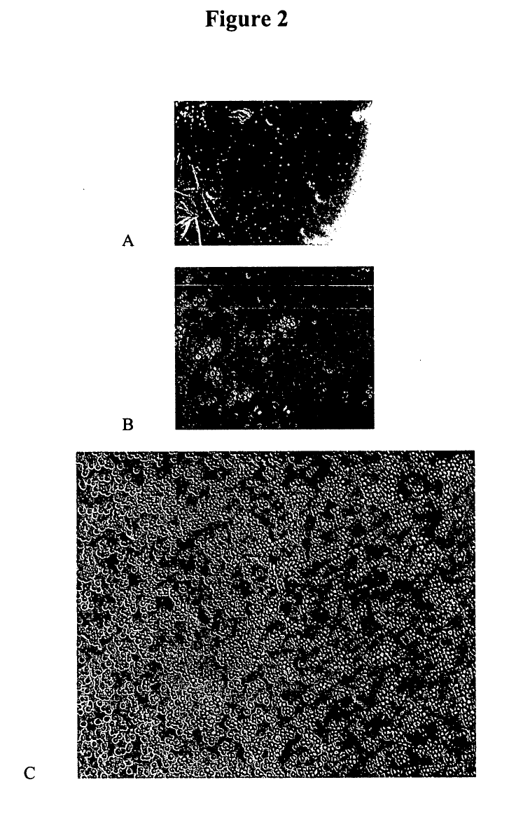

Using the methods of the present invention, a solid or semi-solid matrix of immobilized ligands is provided that can be used to attach living cells and grow tissue. In a preferred embodiment, the ligand-modified biopolymer can be a peptide-agar matrix. The peptide-agar matrix can be used to coat a solid support surface for micropatterning cell adhesiveness (Cass, T. and Ligler, F. S. eds. “Immobilized Biomolecules in Analysis: A Practical Approach”, Oxford University Press, 1998), coat an artificial scaffolding for tissue engineering (Radisic, M. et al. Biotechnology and Bioengineering 2003, 82(4): 403; Ponticiello, M. S. et al. J Biomed. Mater. Res. 2000, 52:246), and form a gel matrix in which a three-dimensional cell culture system can be developed (Lang, S. H. et al. Cell Growth & Differentiation 2001, 12:631). FIG. 2 shows Jurkat cells bound to the surface of a peptide-agar matrix, demonstrating the performance of two-dimensional cell growth on the surface of a peptide-agar matrix.

### V. EXAMPLES

### Example 1

This example illustrates the preparation of a Peptide-Agarose Microarray I

Preparation of ester-linked ketone-modified agarose. In a typical procedure, 1 g of agarose (type XI: low gelling temperature, I in Scheme 1) was melted in 50 mL ddH₂O. The solution was added dropwise into 200 mL of stirred DCM to form agarose beads. The beads or blocks were collected by filtration and washed with acetonitrile. The beads were pressed and cracked into smaller size and dried by lyophilization. The dry, pretreated agarose (0.66 g, calc. 8.8 mmol —OH) was dissolved in 50 mL DMF with heating. A solution of levulinic acid (0.45 mL, 4.4 mmol), DIC (0.34 mL, 2.2 mmol) and DMAP (53 mg, 0.22 mmol) in 30 mL DMF was added into the agarose DMF solution. The mixture was stirred at room temperature overnight (>8 hours). The solution was poured into 500 mL diethyl ether to provide a precipitate. The resulting precipitates were filtered and washed with ether. By controlling the amount of levulinic acid anhydride used in the reaction, modified agarose (II, in Scheme 1) with different loadings of ketone were obtained.

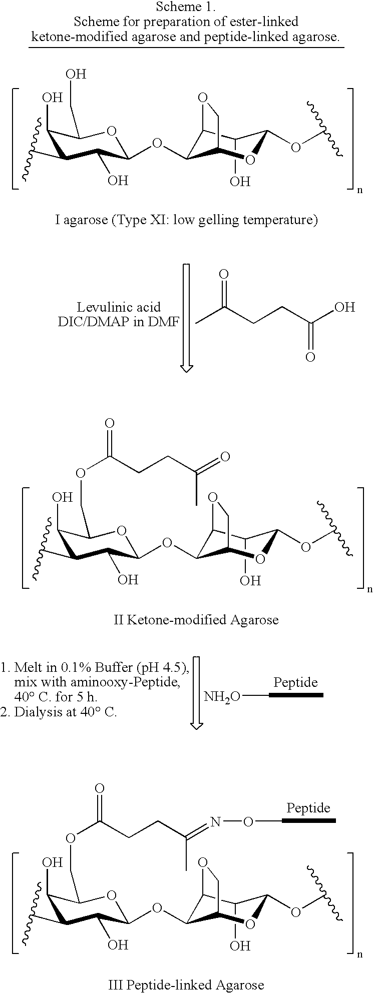

Chemoselective ligation of peptide to the ketone-modified Agarose. In a typical procedure, 10 mg of the ketone-modified agarose (calc. ketone 0.033 mmol) was dissolved in 5 mL of 25% DMSO/acetate buffer (0.05 M NaAc/HOAc, pH 4.5) by gentle heating. A solution of sppLDIn-Tdts-Dpr(Aoa)-NH₂ (45 mg, 0.039 mmol) in 0.5 mL of DMSO/acetate buffer was added. The mixture was stirred at 40° C. for 5 hours. The solution was dialyzed against 5000 mL×4 ddH₂O for three days at 40° C. Peptide-linked agarose (III, in Scheme 1) was obtained as a powder form after lyophilization. Amino acid analysis quantifies the loading of peptide on agarose. pLDIn-Tdts-Dpr(Aoa)-NH₂-linked agarose was also prepared.

Hydrolysis of peptide-agarose conjugate for quantitative amino acid analysis. 5.0 mg dry peptide-agarose conjugate was dissolved in 1.0 mL of 5% formic acid in 50%. acetonitrile, and 100 ∞L of the final mixture was transferred to glass hydrolysis tube and dried. The residue was then treated with 200 μL of 6 N HCl/0.1% phenol at 110° C. for 24 hours, and dried. The residue was dissolved in norleucine (internal standard) dilution buffer to a final volume of 2.5 mL. 50 μL of the sample was injected for quantitative amino acid analysis. The loading of peptide on agarose was determined to be 0.3 mmol g⁻¹.

Microarray application. In a typical procedure, a 50 μL solution of peptide containing an aminooxy group (0.01 mM-0.6 mM) in 25% DMSO/acetate buffer (pH 4.5) is mixed with a 50 μL solution of the ketone-modified agarose (0.2 mg/mL) in 25% DMSO/acetate buffer (pH 4.5). The mixture is incubated at room temperature overnight. Without further purification, the peptide-agarose solution is then spotted onto a pre-cleaned polystyrene or glass slide with a commercially available arrayer (Wittech arrayer 04 (Taiwan)). Spot sizes were about 300 μm in diameter and spotted at 750 to 900 μm intervals (center to center). Multiple samples can be spotted on a large number of slide replicates. After spotting, the slides are transferred to a humidified container for overnight incubation or air-dried for an hour or so, at which point they are ready for subsequent biological assays.

Biotin detection. A printed slide was first rinsed with PBS, and blocked for 1 hour at room temperature with 5% bovine serum albumin (BSA, Fisher) in 1% Tween 20 phosphate buffered saline (PBST, 10 mM Na₃PO₄, pH 7.4, 140 mM NaCl, 1% Tween 20). Streptavidin-horseradish peroxidase conjugate (1/8000 dilution in 1% BSA with PBST, BioRad) was added to the microarray slide, and incubated for 1 hour at room temperature. After thorough washing, 1 mL of enhanced luminol reagent and 1 mL of oxidizing reagent (PerkinElmer Life Sciences, Inc.) were added to the slide, followed by exposure to X-ray film.

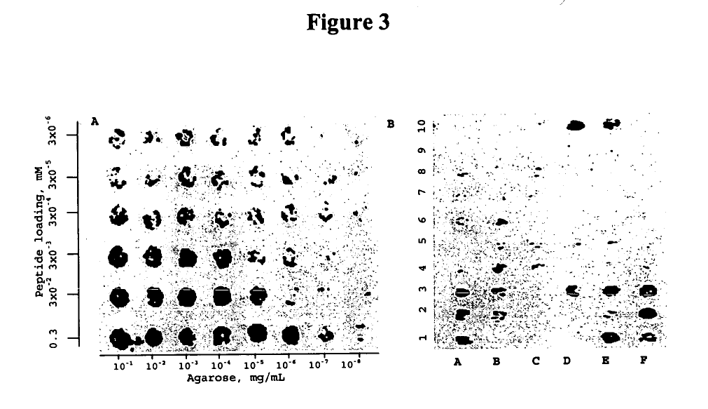

Cell-adherent or cell culture application and micro cell-adhesion assay. Microarrays of 60 different cancer cell-binding peptides were prepared, and evaluated for their ability to bind Jurkat cells in a micro cell-adhesion assay. The results are shown in FIG. 3. For this example, two preparations of peptide-agarose were prepared: low loading (0.02 mmol/g) and high loading (3 mmol/g). They were melted in PBS (1% w/v) and mixed with varying amounts of 1% (w/v) regular agarose solution. The peptide-agarose solution was then added onto a slide and allowed to gel. Microarray slides were first blocked with 5% BSA in PBS for 30 min and then rinsed with PBS. A suspension of T-lymphoma Jurkat cells (obtained from ATCC and grown in 10% FBS in RPMI 1640, 1% penicillin/streptomycin, 1% glutamine at 37° C. and 5% CO₂) was added to the microarray slide. After incubation for 1-2 hours at room temperature, the cell suspension was poured out and the agarose gel surface was washed gently with PBS several times to remove free cells. The microarray slide was then treated with formalin solution (5% in PBS), thoroughly washed with PBS buffer, and stained with 1% violet crystal solution for 1 min. The stained microarray was then directly scanned (UMAX, Astra 2400S). According to FIG. 3A, the spots with modified agarose at 0.1-10⁻⁴ mg/mL and the peptide at 0.3-3×10⁻³ mM shows excellent cell binding. The data suggests that these concentrations are suitable for microarray cell-binding detection. FIG. 3B depicts the binding result of Jurkat cells to 60 different peptide-agarose conjugates. Jurkat cells bind strongly to 10 of these 60 peptides. Background binding is minimal. All these peptide sequences were originally identified via a on-bead cell binding assay of one-bead one compound combinatorial approach (see Falsey, J. R. et al., Bioconjugate Chem. 2001 12:346-353; Park, S. et al. in Peptides: The Wave of the Future (Proceedings of the 2nd International and the 17th American Peptide Symposium, M. Lebl and R. A. Houghten (Eds),), San Diego, Calif., United States, 9-14 Jun. 2001, Kluwer Academic Publishers, Dordrecht, 2002, pp. 180-182). Therefore the use of peptide microarrays can be used to validate the binding of cells to peptide sequences identified through OBOC screening methods.

### Example 2

This example illustrates another method for the preparation of a Peptide-Agarose Microarray II.

Preparation of amide-linked ketone-modified agarose.

Oxidation of agarose. In a typical procedure, 3.28 g of agarose (type XI: low gelling temperature, I in Scheme 2) is melted in 5 g of sodium carbonate solution in 250 ml ddH₂O. Into the solution, 30 mg of TEMPO is dissolved in 1 mL of DMSO and 0.2 g of potassium bromide. The pH value is adjusted to 10-11 by 1M NaOH solution. While stirring 4.0 mL of 1.3M sodium hypochlorite solution is added drop-wise into the solution. The reaction is stirred for 4 hours. The insoluble byproduct is filtered away and the filtrate is treated with 3 fold excess of ethanol to precipitate the product. The filter cake is washed with 70% ethanol (3×10 mL) and dried by lyophilizer.

Ketone-modification of oxidized agarose. 1 g of oxidized agarose (II in Scheme 2), 0.5 mL of 1,3-diaminopropane, 30 μL of DIC and 40 mL of DCM is added in a flask. The suspension is stirred overnight. The product is collected by filtration and washed with DCM and ethanol. The sample is then evaluated by the Chloranil test, indicating the presence of amino groups. Subsequently, 0.5 g of the resulting white powder, Ig of N-hydroxy succinimidyl levulinate, 0.2 mL of DIEA and 40 mL of DCM is mixed and stirred overnight. The solution is filtered away and the residue washed with DCM (3×10 mL) and ethanol (3×10 mL). The product (ketone-modified agarose, III in Scheme 2) is then evaluated by the Chloranil test, indicating the absence of any amino groups.

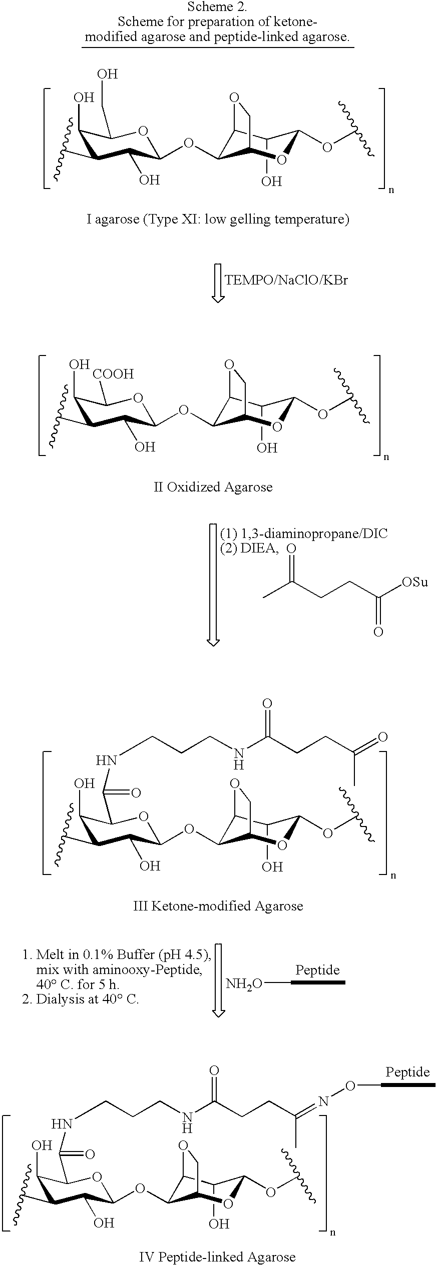

Chemoselective ligation of ligands to the ketone-modified agarose. In a typical procedure, the ketone-modified agarose is dissolved in 5 ml of 50% DMSO/acetate buffer (0.05 M NaAc/HOAc, pH 4.5). A solution of pre-made ligand-Dpr(Aoa)-NH₂ (slight excess) in 0.5 ml of DMSO/acetate buffer is added. The mixture is stirred at room temperature overnight. The solution is dialyzed against 5000 ml of double distilled water for three days. Peptide-linked agarose (IV in Scheme 2) is obtained in a powder form after lyophilization.

Preparation of Microarrays. In a typical procedure, a solution of peptide containing an aminooxy group in 50% DMSO/acetate buffer (pH 4.5) is mixed with a solution of the ketone-modified agarose in 50% DMSO/acetate buffer (pH 4.5). The mixture is incubated at room temperature overnight. Without further purification, the peptide-agarose solution is then printed onto a glass slide with a commercially available arrayer. Thousands of samples can be spotted on one slide and a large quantity of slide replicates can be easily produced. After spotting, the slides are air-dried overnight for subsequent biological assays.

Protein and cell extract labeling. Commercially available c-src PTK (UBI, Lake Placid, N.Y.), Etk and Brk PTKs are first dialyzed with PBS to remove any free amine in the sample (final sample volume 100 μl). The proteins are then labeled with Cy-3-NHS or Cy-5-NHS (Molecular Probe) according to the protocols provided by the manufacturer. For whole cell extracts, the parent cell line and the Brk transfected cell line were lysed separately with standard lysis buffer containing a non-ionic detergent, and protease inhibitors. The cell extracts are then labeled with Cy-3 or Cy-5 according to the protocols provided by the manufacturer.

Microarray analysis. The fluorescent-labeled proteins and cell extracts are diluted serially with PBS/Tween (pH7.0) and layered over the microarray and incubated for 1 hour. Bound fluorescent-labeled proteins are analyzed with a fluorescent scanner. Different fluorophores can be used to label cell extracts from different cell populations. Each slide is scanned on a 2-color fluorescent scanner from General Scanning (ScanArray 3000), creating a 16-bit TIFF image. The slide is scanned by a laser focused to 10 μm of the glass surface. The images are downloaded from the scanner and analyzed using a commercial software program (ImaGene from BioDiscovery, Santa Monica). The program uses a deformable template/blop detection algorithm to detect and surround each data spot and automatically detects the regions of fluorescent signals, determines signal intensity, performs statistical analysis, and compiles the data into an Excel spreadsheet for further analysis. GeneVision from BioDiscovery is used to mine the data and provide visualization tools (2-D and 3-D scatter plots, interactive ratio histogram plotting, hierarchical and neural network clustering, Principal Component Analysis, and Time Series Analysis). Cell extracts can be tested individually or mixed together to determine if there is a difference between binding of proteins contained in the cell extracts of normal and tumor cells.

### Example 3

This example illustrates another method for the preparation of a Peptide-Agarose Microarray.

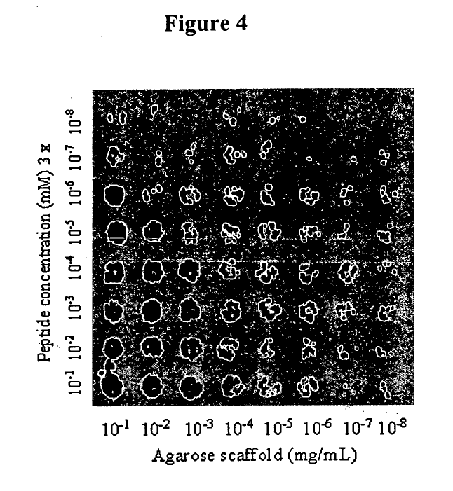

Preparation of peptide-agarose conjugate microarray. In this example, peptide-agarose conjugate microarrays were prepared using various amounts of aminooxy-peptide and ketone-agarose scaffold The concentration of agarose ranged from 0.1 to 10⁻⁸ mg mL⁻¹ and the peptide concentration was varied from 0.3 to 3×10⁻⁸ mM. Sixty-four solutions of sppLDIn-agarose conjugate were prepared and spotted on both glass and polystyrene slides. After microarray spotting, the spotted slides were air-dried and the micro cell-adhesion assay performed. According to the results (FIG. 4), the spots with ketone-agarose at 0.1-10⁻⁴ mg mL⁻¹ and the aminooxy-peptide at 0.3-3×10⁻³ mM showed excellent cell binding. At the highest agarose concentration (0.1 mg mL⁻¹, excellent cell binding was still observed even with the peptide concentration at 3×10⁻⁶ mM. This corresponds to a ratio between 0.1 equiv of peptide to ketone group on agarose. At the highest peptide concentration (0.3 mg mL⁻¹), excellent cell binding was still be obtained with the agarose concentration approximately 10-3 mg mL⁻¹.

### Example 4

This example illustrates another method for the preparation of a small molecule library.

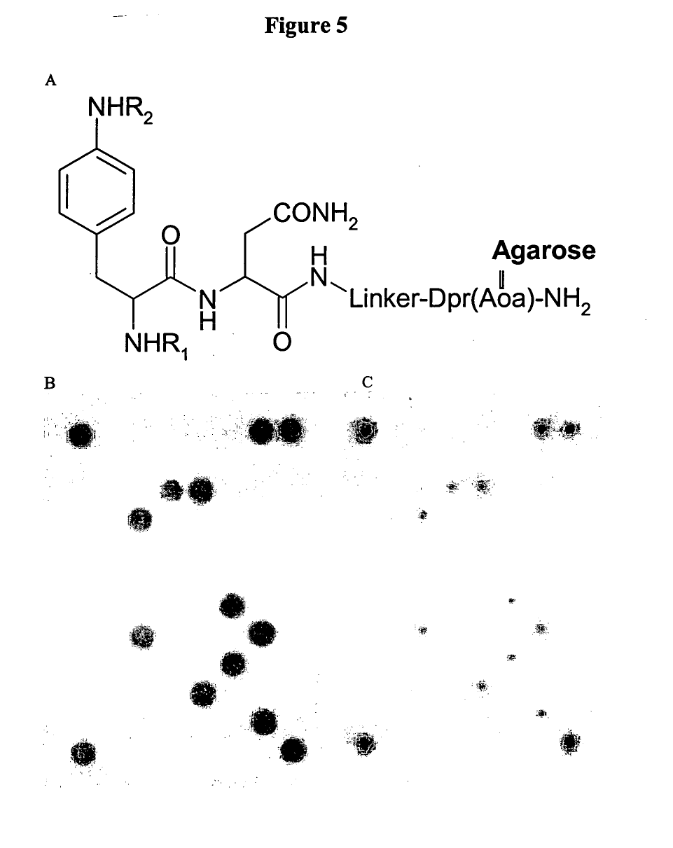

Small molecule microarrays for Streptavidin. The above mentioned one-aggregate one-compound method was used for the synthesis of a 25 member encoded small molecule library with two random positions: R1 and R2. The chemical structure of library is shown in FIG. 5A. The α-amino group of a p-nitrophenylalanine was first acylated with 5 different carboxylic acids including d-biotin. The aromatic nitro group was then reduced with SnCl₂ and acylated with 5 other carboxylic acids, as described in Liu, R. et al. JACS 2002, 124: 7678. The functional group R1 attached on α-amino group of the scaffold is encoded by amino acid Aaa₁ and the second building block R2 is encoded with Aaa₂ on the black colored beads. The compounds of the library were cleaved from bead aggregates and conjugated to ketone modified agarose via an oxime linkage (Scheme 2). The residual bead aggregates were washed and stored for subsequent decoding. The library of ligand-agarose conjugates was then printed on a PVDF membrane with an automatic microarrayer using a 300 μm needle with 900 μm spot distance (FIG. 5B) and a 100 μm needle with 400 μm spot distance (FIG. 5C). The microarray was then incubated with streptavidin-alkaline phosphatase complex for one hour, washed, and incubated with BCIP substrate for one hour to yield blue color spots (FIGS. 5B and C). To facilitate final orientation and alignment of the microarrays, the four corners were marked with d-biotin-agarose conjugate. In addition, the top-right corner was marked with two adjacent spots. The corresponding encoding beads were isolated from the stored bead aggregates (mother plate) and submitted for Edman-based sequencing analysis. Upon decoding, the 9 additional stained spots (FIG. 5B) were all found to have d-biotin at the R1 position, but there is no significant preference for R2. In another experiment, a similar microarray approach was used to print a number of different cell surface binding peptides on polystyrene slides and demonstrated that differential adhesion of intact cells to these peptide microarrays can be detected.

This simple experiment clearly demonstrates the feasibility of combining the one-aggregate one-compound method with the microarray technology of the present invention, for rapid screening of large number of small molecule compounds for biological activites. Because excess ligands, but equal amounts of ketone-modified agarose, are used in each of the chemoselective ligation reactions, the final concentration of covalently linked ligands are identical in each microarray spot. This last but very important feature is unique for this microarray platform, leading to its wide utility by investigators in the fields of proteomics and diagnostics.

### Example 5

This example illustrates the preparation of a Peptide-Protein Conjugate and a strategy for the preparation of chemical microarrays using macromolecular scaffolds.

Human serum album (HSA) contains around 60 ε-amines from Lys residues that allow chemical modification. By use of N-succinimidyl levulinic acetate HSA can be readily modified with ketone groups with a preferred loading. The ketone-modified HSA can be used for conjugation of any synthetic peptide or small molecule containing an aminooxy group. The conjugation takes place at the ketone moiety of the modified HSA and the aminooxy group of the synthetic compound giving the oxime linkage. In this reaction, other amine groups in the synthetic compound or in the HSA will not react with the ketone group in HSA. Scheme 3 shows the strategy of preparation of the ketone-modified HSA (I, in Scheme 3) and subsequent chemoselective ligation of chemical compounds (drug) to the scaffold.

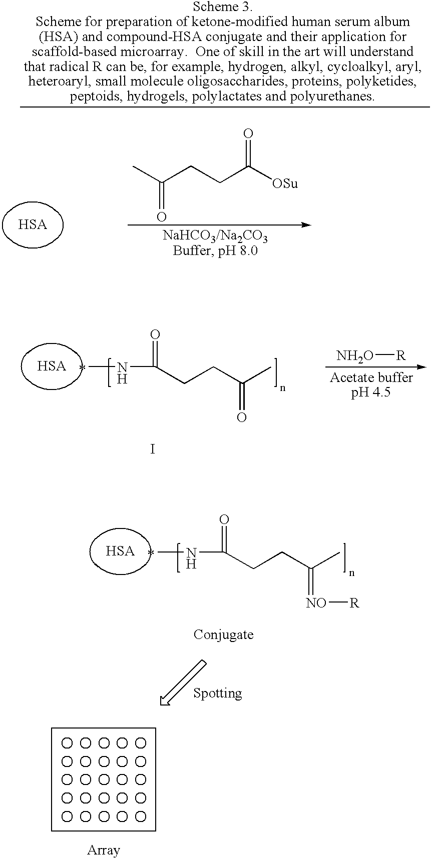

The macromolecular scaffolds are first functionalized with ketone groups and compounds of interest containing and aminooxy group are conjugated onto the ketone-modified scaffolds through a chemoselective oxime ligation. The conjugate mixtures are the spotted directly onto a plastic or glass surface to form compound microarrays. Because a constant amount of scaffold is used in the presence of excess compound in the ligation reaction, the amount of compound actually immobilized per microarray spot is constant and dependent on the scaffold concentration. Using this approach, 60 different peptides were ligated to human serum albumin or agarose scaffolds, and the peptide conjugates subsequently printed on glass or polystyrene surface to form microarrays. These peptide microarrays were subsequently evaluated and optimized for binding of Jurkat leukemic cancer cells.

Peptide synthesis. Sixty peptides known to bind specifically to many different cancer cell lines (see Aina, O. H. et al. Biopolymers 2002, 66:184-199) were selected and their aminooxy derivatives were prepared for scaffold ligation and microarray application. Peptides were synthesized by standard solid phase peptide synthesis via Fmoc-chemistry on Rink Amide Resin (Scheme 4). Reagents for peptide synthesis were purchased from Advanced ChemTech, Louisville, Ky. or Chem-Impex International, Wood Dale, Ill. Fmoc-4,7-dioxa-1,10-decanediamine, the Fmoc protected hydrophilic linker, was prepared according to Song, A. et al. Bioorg. Med. Chem. Lett. 2004, 14(1): 161-5. Parallel synthesis was performed using a 42-reactor MULTIBLOCK synthesizer (CSPS Pharmaceuticals, Inc., San Diego). All couplings were conducted by HOBt/DIC (Aldrich, Milwaukee, Wis.) activation in DMF (v/v). 25% piperidine in DMF was used to remove the Fmoc group.

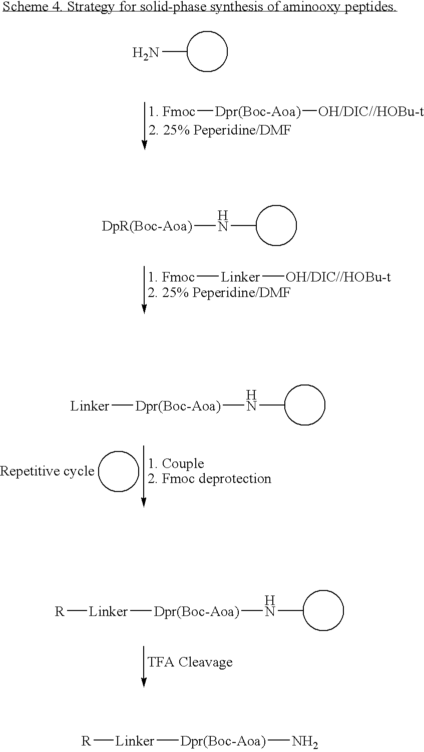

Fmoc-Dpr(Boc-Aoa) was first anchored on to the solid support, followed by incorporation of a hydrophilic linker. Peptide chains or biotin were then assembled on this Linker-Dpr(Boc-Aoa)-resin. In this way, all final products contained a linker (spacer) and a Dpr(Aoa) residue (aminoxy conjugation moiety) at their C-terminus. Aminooxy compounds were cleaved from resin by reagent K (2 hours). Cleavage scavengers were removed by ether-precipitation, and washing of the peptides. By multiple ether-precipitation, all crude products were 70-90% pure as determined by reverse phase HPLC (Beckman) and electrospray mass spectrometry (ESMS). Biotin-Linker-Dpr(Aoa)-NH₂ was cleaved from resin by 95% TFA and precipitated with hexane/ether (2:1). Some of the peptides contain D-cysteines at both termini of the peptide and cyclization via intra-molecular disulfide bridge was achieved in solution by oxidation with DMSO/sodium acetate buffer (1:1) (pH 6.0, overnight).

Preparation of aminooxy-biotin. Biotin was coupled onto Linker-Dpr(Aoa)-Rink Amide Resin by HBTU/DIEA. A solution of 95% TFA (containing 5% water) was used to cleave the product. After precipitation with hexane/ether (2:1), the crude was analyzed with reverse phase HPLC, which indicated the purity at ˜90%. The correct identity was confirmed by electrospray mass spectrometry (ESMS).

Preparation of N-succinimidyl levulinic acetate. 1.24 mL (10.0 mmol) levulinic acid and 1.16 g (10.0 mmol) HOSu were added to 30 mL DMF/DCM (1:5) and the mixture was stirred and cooled in an ice-bath. While stirring, a solution of 2.06 g (10.0 mmol) of DCC in 10 mL DCM was added to the mixture. The final solution was stirred at 4° C. overnight. The suspension was filtered and washed with H₂O. The organic layer was separated and dried over Na₂SO₄. Solvents were removed by evaporation under vacuum. Crystalline product was obtained after treatment with ether. HPLC analysis: single peak (>99%). H¹-NMR: 2.20 ppm (3H, 1 CH₃, s), 2.80-2.89 ppm (8H, 4 CH₂, m).

Preparation of HSA scaffold. In a typical approach, ketone-modified HSA was prepared by acylation of a number of the lysyl-ε-amino groups of the protein with the preformed cross-linking reagent, N-succinimidyl levulinic acetate. HSA (approx. 10 μmol, Sigma Chemical, St. Louis, Mo.) was dissolved under 0-5° C. in 5 mL of 0.1 M NaHCO₃/Na₂CO₃ buffer (pH 8.0). A solution of N-succinimidyl levulinic acetate (50 μmol) in 0.5 mL of DMSO is then added to one portion of the protein solution, although different molar ratios of the cross linker (5, 10, and 300 equiv, relative to the protein) can also be used. The mixture is stirred overnight at room temperature. The reaction is acidified to pH 6.0 and subjected to dialysis (MW cutoff 15000, dm 29 mm, Spectrum Laboratories, Inc., Calif.) against 5 L H₂O (0-5° C., 48 hours). The solution after dialysis is lyophilized affording a white powder.

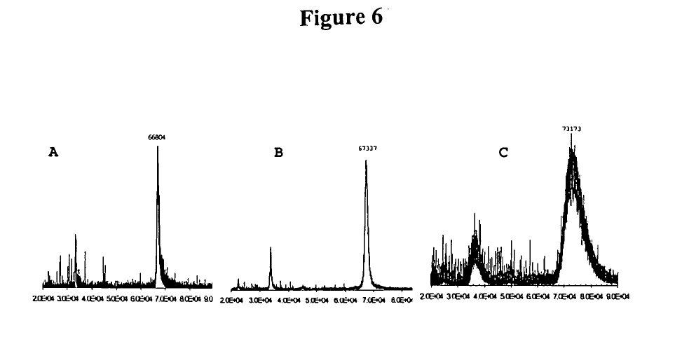

Application the ketone-modified HSA for preparation of protein conjugates. The actual ketone loading was determined by a two-step procedure: first conjugating a synthetic aminooxy peptide to the ketone modified protein, followed by MALDI mass analysis. To a 0.1 mL of 1 mg/mL solution of I (Scheme 3) is added a solution of 0.1 mL of 0.1 mM Dpr(Aoa)-compound (sppLDIn-Tdts-Dpr(Aoa)-NH₂) in NaAc/AcOH buffer (pH 4.5). DMSO was added to the buffer to facilitate the chemical reaction. The mixture is stirred for at least 5 hours at room temperature. The conjugation solution is subjected to extensive dialysis (5 L×3 ddH₂O) and subsequent lyophilization, affording a white powder. FIG. 6 shows the mass spectra of HSA (A), the ketone-HSA (B) and the peptide-HSA conjugate (C). The broad peaks of mass signal are due to heterogeneous nature of the HSA protein with different posttranslational modifications (see below). The peaks of the three HSA preparations are fairly symmetrical. The ketone-HSA scaffold has an average molecular weight of 67 337 (FIG. 6B). Based on the molecular weight of 66 804 for the unmodified HSA (FIG. 6A), the mass shift is about 533 units, which corresponds to approximately 5.4 units of cross linker per protein molecule. The average molecular weight for peptide-HSA conjugate was 73 173±400 Da (FIG. 6C), with an average mass shift of 5836 relative to the ketone-HSA scaffold. This corresponds to 5.2 peptide units per protein. Both values compare favorably with a theoretical loading value of 5.0. The results thus have indicated that the acylation of HSA with N-succinimidyl levulinic acetate and subsequent ligation of peptide occurred quantitatively.

Preparation of biotin-HSA conjugate. The biotin-HSA conjugate was prepared by using the aminooxy-biotin (e.g. biotin-Linker-Dpr(Aoa)-NH₂ purity ˜90%) and the ketone-HSA with the same procedure for preparation of peptide-HSA conjugates.

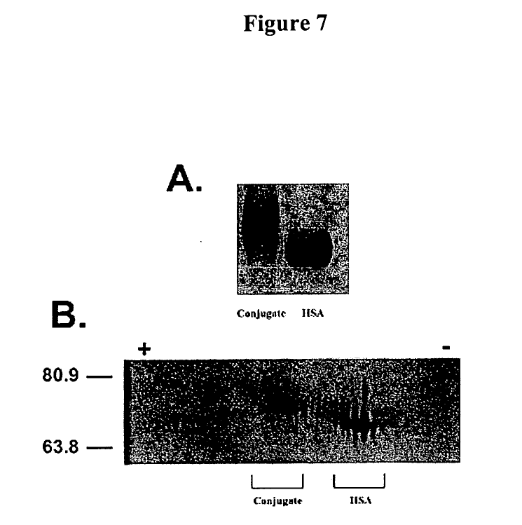

SDS PAGE and 2D PAGE. Unmodified and modified HSA were analyzed using one-dimensional SDS (SDS PAGE) and two-dimensional polyacrylamide gel electrophoresis (2D PAGE). The 10% SDS PAGE was performed using a Protein II (BioRad). The second dimensional PAGE (2D PAGE) was performed using pH 3-10 IPG strips and the MultiphorII (Amersham Pharmacia) for isoelectric focusing in the first dimension and the Protean II (BioRad) (10%) in the second dimension (according to the Amersham Pharmacia 2D PAGE instruction manual). The 1D and 2D PAGE were stained with colloidal Coomassie blue (InVitrogen) and destained in distilled water. Gels were scanned using the Personal Densitometer (Applied Biosystems). FIG. 7 shows the SDS polyacrylamide gel electrophoresis (SDS PAGE) analysis of HSA and peptide-HSA conjugate. As expected, in the one-dimensional PAGE (10% SDS PAGE), the peptide-HSA conjugate migrated as a broader protein band with slightly higher molecular weight than the unmodified HSA (FIG. 7A). 2D PAGE separates proteins by their isoelectric point in the first dimension and molecular weight in the second dimension. HSA and peptide-HSA conjugate samples were resolved on separate 2D gels and the results overlaid (FIG. 7B). Un-conjugated HSA appeared as six discrete spots with slightly different isoelectric points. These spots reflect a variation in posttranslation modifications among different HSA molecules. Peptide-HSA migrated at a slightly higher molecular weight and was more acidic that un-conjugated HSA when analyzed by 2D PAGE. This is consistent with the mass spectra data (FIG. 6C), which showed the addition of five peptides to each HSA molecule. These changes in mass and charge result from the addition of five acidic residues, contributed by the aspartate in the sppLDIn peptide, which leads to a decrease in the isoelectric point of the final peptide-HSA conjugates and an increase in molecular weight (FIG. 7B).

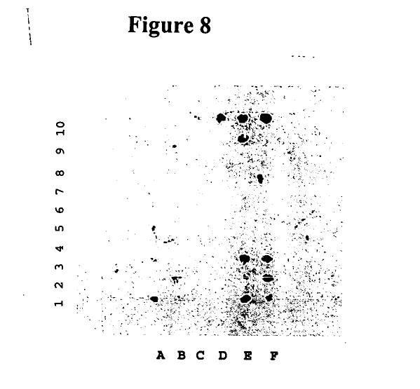

Microarray application. To evaluate the binding property of the peptide-HSA conjugate to Jurkat cells, the conjugate was spotted onto a polystyrene slide. In a typical procedure, a 50 μL solution of peptide containing an aminooxy group (0.1-20 μM) in 25% DMSO/acetate buffer (pH 4.5) is mixed with a 50 μL solution of the ketone-modified HSA (0.2 mg/mL) in 25% DMSO/acetate buffer (pH 4.5). The mixture is incubated at room temperature overnight. The solution then is printed onto a plastic slide. Using this procedure, a number of samples are made and printed on slides by an automated arrayer. After spotting, the slides are incubated in a moisturized chamber at room temperature for 5 hours or longer. The slides are then ready for biological assays. Microarrays of 60 different cancer cell-binding peptides were prepared, and evaluated for their ability to bind Jurkat cells in a micro cell-adhesion assay. The peptide arrays were overlaid and incubated with Jurkat cells for 30-60 min. The free cells were then gently removed and the bound cells fixed with formalin solution and stained by crystal violet. FIG. 8 shows the assay results of Jurkat cell binding on a spotted slide that contained the 60 peptide-HSA conjugate spots. The binding assay has shown 9 peptides that Jurkat cells bind most strongly. Most strong binding spots in peptide-HSA microarray were also observed in the agarose approach.

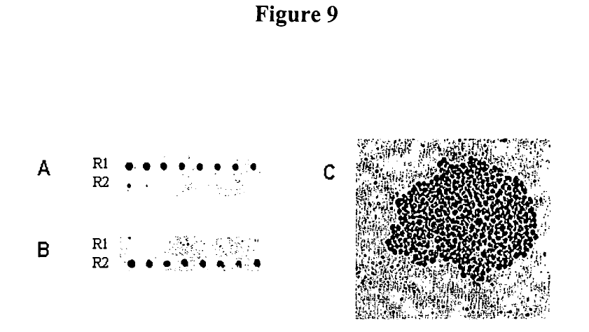

In FIG. 9, two assays were performed on two duplicate slides (A and B). Each slide has 8 spots of biotin-HSA conjugate (top row, R1) and 8 spots of sppLDIn-HSA conjugate (bottom row, R2). Slide A was incubated with avidin-horseradish peroxidase (HRP) and then HRP substrate; and slide B was overlaid with Jurkat cells, washed, and stained (see methods). Slide A showed staining of the 8 spots of biotin-HSA, whereas the spots with the sppLDIn-HSA conjugate showed no staining demonstrating that only biotin-HSA could be detected. In contrast, in slide B, only the 8 spots of sppLDIn-HSA were stained, indicating that Jurkat cells were bound only to the sppLDIn-HSA conjugate spots and not to the biotin-HSA spots.

### Example 6

This example illustrates another method for the preparation of a Peptide-HSA Microarray.

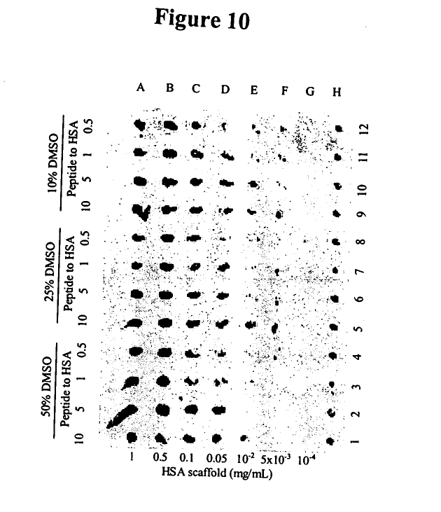

Preparation of peptide-HSA conjugate microarray. In this example, peptide-HSA conjugate microarrays were prepared using various amounts of aminooxy-peptide, ketone-HSA scaffold, and DMSO. After spotting, the slides were stored in a humidified container overnight to allow conjugate physically absorbed on the surface. The incubation time can be shorter (e.g. 2˜3 hs) and the humidified conditions may not be required. The slide was then blocked with 5% BSA solution (FisherChemical, Fair Lawn, N.J.) and the Jurkat micro cell-adhesion assay performed as in Example 1. Results from the assay indicated (FIG. 10) that the spots with modified HSA at 0.1-1.0 mg mL⁻¹ and the peptide at 0.5˜10 equivalent (relative to HSA-ketone) showed excellent cell binding. No appreciable difference in results were observed for spots using 10-50% DMSO.

### Example 7

This example illustrates the use of a microarray wherein a small organic molecule is attached to the peptide conjugate to assay for reactivity against non-peptide specific antibodies.

Source of Antibodies. The production and specificity of murine anti-PDC monoclonal antibodies, clones 2H4, C355.1 and 4C8, has been previously described (Migliaccio, C. A. et al. J. Immunol. 1998, 161: 5157). Anti-influenza hemagglutinin (HA) antibodies were obtained from Roche Applied Science (Indianapolis, Ind.). Sera from the 6-bromohexanoate-BSA and BSA immunized rabbits were obtained as previously described (see Leung, P. S. et al. J Immunol. 2003, 170:5326).

Rabbit Immunization. Female New Zealand white rabbits at 16 weeks of age were immunized subcutaneously with 100 μg/animal of 6-bromohexanoate-BSA (n=10) or 100 μg/animal of BSA alone (n=8) incorporated in Freund's complete adjuvant and then boosted subcutaneously every 2 weeks with the same dose of antigen in Freund's incomplete adjuvant. Sera were collected 8 weeks after initial immunization and every 4 weeks thereafter for 22 months for analysis of AMA reactivity using the high throughput xenobiotic-peptide-agarose assay described below. Animal protocols were approved by the Institutional Review Board of the University of California at Davis.

Peptide Synthesis. Four peptides (influenza hemagglutinin (HA) peptide YPYDVPDYA; PDC peptide DKATIGFEVQEE; mutant PDC peptide AKATIGFEVQEE; and the bovine serum albumin peptide; FKGLVLIAFSQY) were synthesized on Rink Amide MBHA Resin (GL Biochem, Shanghai) (see Fields, G. B. and Noble, R. L. Int. J. Pept. Protein Res. 1990, 35:161). Briefly, first, Fmoc-Dpr(Boc-Aoa) (Novabiochem, Switzerland) (see Wahl, F. and Mutter, M. Tetrahedron Letters 1996, 37:6861) was attached to the solid support followed with the hydrophilic spacer (N-Fmoc-2,2′-(ethylenedioxy)bis(ethylamine)monosuccinamide) (see Song, A. et al. JACS, 2003, 125:6180) and the appropriate amino acid sequence. Amino acid coupling was conducted by a three-fold molar excess of Fmoc protected amino acid, HOBt/DIC activation in DMF until Kaiser test (see Kaiser, E. et al. Analytical Biochemistry 1970, 34:595) was negative. The Fmoc protecting group was removed by 20% piperidine in DMF (30 min). After removal of the Fmoc group from the last residue (Asp), the N-terminal amino group was acylated with acetic anhydride and DIEA. A mixture of TFA, TIS and ddH₂O (95:2.5:2.5 v/v/v) was applied to cleave compounds from the resin and remove the side chain protecting groups. Peptides were then purified by preparative C-18 reversed phase (Vydac, Hesperia, Calif.) HPLC to yield >95% purity.

Modification of agarose and conjugation to peptide. In a manner similar to Example 1, one gram of agarose (type XI: low gelling temperature (Sigma, St. Louis, Mo.)) was melted in 50 ml ddH₂O. The agarose solution was added dropwise into 200 ml of stirred DCM to form agarose beads. The beads or blocks were collected by filtration, washed with acetonitrile, crushed into smaller pieces (<5 mm) and lyophilized. The pretreated dry agarose (0.66g, calc. 8.8 mmol —OH) was then dissolved in 50 ml DMF with heating. A solution of levulinic acid (0.45 ml, 4.4 mmol), DIC (0.34 ml, 2.2 mmol) and DMAP (53 mg, 0.22 mmol) in 30 ml DMF was added to the agarose (in DMF) solution. The mixture was stirred at room temperature overnight (>8 hours). The solution was poured into 500 ml diethyl ether. The resulting precipitates were filtered and washed with ether. Ten ml of this modified agarose solution (5 mg/ml) was added to 10 ml of the appropriate peptide solution (20 μM) in a 0.05 M NaAc/AcOH buffer (pH 4.5) containing 50% DMSO. The mixture was stirred for 5 hours at 65-70° C. Ketones on modified agarose react selectively with amino-oxy groups on peptides to form oximes at slightly acidic pH (see Lemieux, G. A. and Bertozzi, C. R. Trends Biotechnol. 1998, 16:506; Shao, J. and Tam, J. P. JACS 1995, 117: 3893). The conjugation solution was subjected to dialysis and subsequently lyophilized. Loading of each peptide was calculated by a quantitative ninhydrin test at 570 nm and was determined to be: PDC peptide=95.5 μmol/g, mutant PDC peptide=83.5 μmol/g and albumin peptide=81.5 μmol/g.

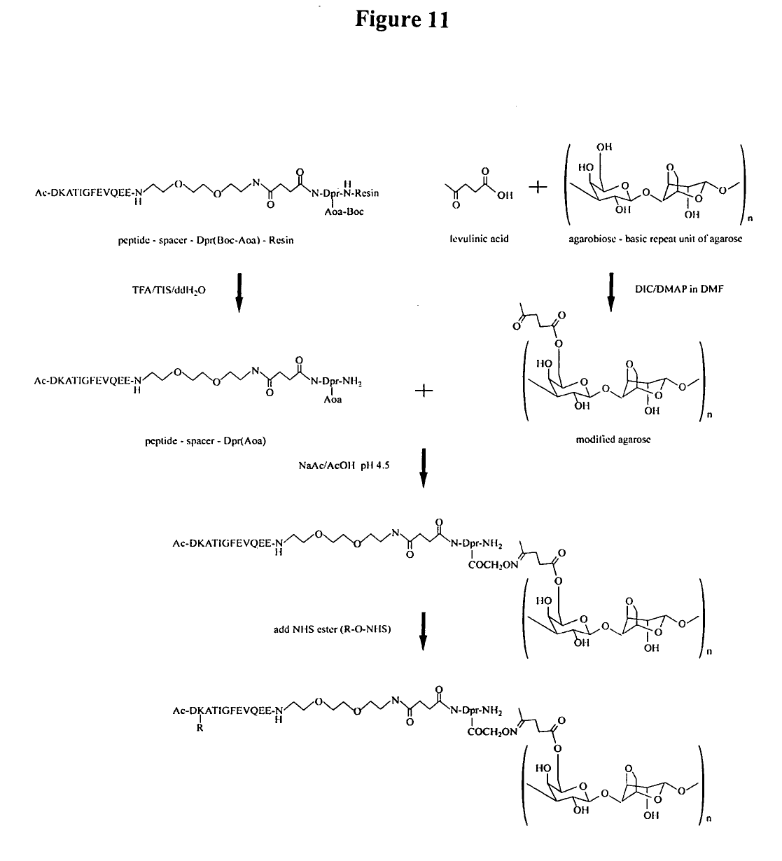

Synthesis of mimeotopes and coupling with peptide-agarose conjugate. Carboxylic acid derivatives of twenty-three xenobiotic compounds were used in this study. Compounds 1-19 were synthesized as described in Long, S. A. et al. J. Immunol. 2001, 167:2956 which is herein incorporated by reference in its entirety. The carboxylic acid derivatives are then reacted with NHS to form the corresponding NHS esters. These 23 compounds, in addition to lipoic acid with NHS ester, were coupled to the lysine residue on the PDC-E2 peptide-agarose conjugate as follows. Briefly, 0.4 mg of the PDC-E2 peptide-agarose conjugate and 10 μmol of each of the NHS esters were mixed in 40 μl of DMSO. Mixtures were incubated at room temperature for 2 hours. To ensure complete coupling, a quantitative ninhydrin test at 570 nm was performed. A schematic representation of the conjugation chemistry is shown in FIG. 11.

Preparation of microarray and analysis of immunoreactivity. Xenobiotic compounds-peptide-agarose mixture were diluted (0.1%) in 0.1 M Na₂CO₃/NaHCO₃ buffer (p)H 9.0), and transferred to 96 well plates. Thereafter mixtures were spotted onto glass slides (Mercedes Medical, Florida) using the Affymetrix 417 Microarrayer (Affymetrix, Santa Clara, Calif.). Each sample was spotted in triplicate, with a spot size of 150 μm in diameter. Spotted microarrays were stored at 4° C. until use. Before use, microarrays were blocked with 3% non-fat dry milk in PBS buffer for 1 hour at room temperature, and individual slides were thereafter incubated with diluted antibody samples (rabbit sera 1:250, murine anti-PDC monoclonal antibody 1:1) in 1 ml of blocking buffer (3% non-fat dry milk in PBS with 0.05% tween-20) (PBST) for 1 hour at room temperature. After thorough washes with PBST, 1 ml of the Cy3 conjugated secondary antibody (1 μg/ml) (Zymed Laboratories Inc. San Francisco, Calif.) in blocking buffer was added to each slide and incubated at room temperature for 30 min. Subsequently slides were washed in PBST for 10 min and in water for 15 sec. Arrays were then dried and scanned using the Affymetrix 428 Array Scanner. To validate peptide microarray sensitivity, four different concentrations (0.1%, 0.03%, 0.01% and 0.004%) of the control HA peptide were spotted. Serially diluted anti-HA monoclonal antibodies (1000 ng/ml, 167 ng/ml, 28 ng/ml, and 5 ng/ml) were assayed individually. Data analysis was performed utilizing the ImageQuant software (Molecular Dynamics, Sunnyvale, Calif.) (Christ, S. A. et al. Electrophoresis 2000, 21:874). Mixtures of xenobiotics and agarose were also spotted and analyzed as controls. To derive net reactivity against xenobiotics coupled with peptide back bone, the mean intensity of reactivity of the experimental rabbit sera against the mixture of xenobiotics and agarose was subtracted from the mean intensity obtained on the corresponding peptide coupled with xenobiotics or lipoic acid. Statistical analysis was performed using JMP software (SAS Institute Inc. NC). Paired “t” test was performed to compare differences of the signal intensity between pre- and post-immunized sera.

Comparison between ELISA and microarray assay. Rabbit sera (n=5) at one month post-immunization were serially diluted (1:250, 1:750, 1:2250, 1:6750 and 1:20250) and IgG reactivity to small molecule-peptide-agarose conjugates were determined by both the ELISA and microarray assay. Briefly, ELISA plates were coated with 50 μl of each individual xenobiotic compounds-peptide-agarose mixture in DMSO (1 mg/ml) for 2 hours at room temperature, then antigens were removed and plates dried overnight at room temperature. Dried ELISA plates were thereafter blocked with 3% non-fat dry milk in PBS and incubated with serially diluted rabbit sera for 1 hour at room temperature. After washing, the plates were incubated with HRP conjugated mouse anti-rabbit IgG (Zymed, San Francisco, Calif.) antibodies for 30 min at room temperature, washed and incubated with 2,2′-azino-bis(3-ethylbenzthiazoline-6-sulfonic acid) containing hydrogen peroxide (Kirkegaard & Perry Laboratories, Inc, MD). Likewise, IgG reactivity of rabbit sera at 0, 1, 6, 12 and 22 months post-immunization against recombinant human PDC-E2 protein (see Leung, P. S. et al. J. Immunol. 2003, 170:5326) and KLH (Sigma, St. Louis, Mo.) was determined at 1:100 sera dilution by standard ELISA.

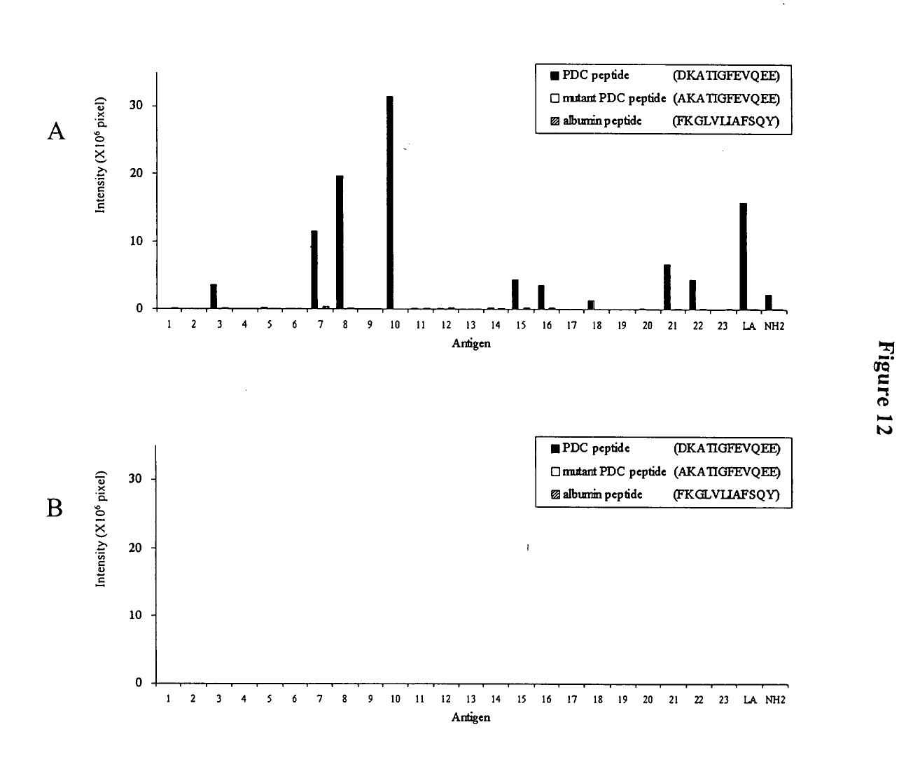

Detection of antibody against lipoic acid and xenobiotics. The unique xenobiotic-peptide-agarose microarray platform was used to screen for the fine specificity of the binding of anti-PDC-E2 antibodies with an emphasis on identifying structures that mimic the molecular image formed by the association of lipoic acid with the immunodominant PDC-E2 peptide. Various peptide backbones, including PDC peptide, mutant PDC peptide or albumin peptide were each coupled with each of the twenty-three xenobiotics and lipoic acid. Reactivity of three different murine anti-PDC monoclonal antibodies was also studied. Monoclonal antibody, 2H4, bound strongly to 6-bromohexanoate, compounds 8, 10 (for numbering of compounds see Long, S. A. et al. J. Immunol. 2001, 167:2956) and lipoic acid on the PDC peptide (FIG. 12A). Weak reactivity to compounds 3, 15, 16, and 18 (for numbering of compounds see Long, S. A. et al. J. Immunol. 2001, 167:2956), and the non-lipoylated native PDC peptide were also detected. 2H4 did not react to xenobiotics conjugated to other peptides. Interestingly, clone 4C8 or C355.1, which are other murine monoclonal antibodies against PDCE2, did not react to any of these xenobiotics. As noted, the normal murine IgG did not react to any of the xenobiotic conjugates (FIG. 12B), including lipoylated peptide or the peptide alone demonstrating the specificity of the binding of the 2H4 antibody.

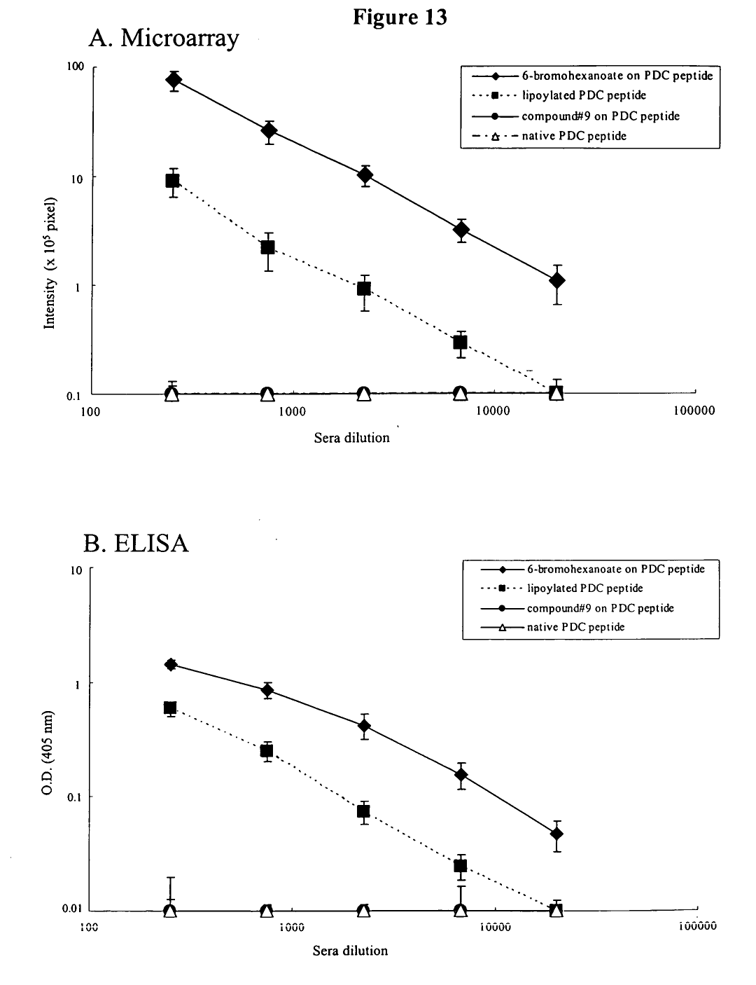

Validation of peptide microarray assay. To validate the microarray assay, data obtained using the microarray and ELISA were compared. Rabbit sera (n=5) at one month post-immunization with human recombinant PDC-E2 were serially diluted and their IgG reactivity against 6-bromohexanoate, compound 9, lipoic acid on PDC-E2 peptide and non-lipoylated native PDC-E2 peptide were determined by both ELISA and microarray assay (FIG. 13). Both results demonstrated a dilution dependent response with sera at a dilution of 1:6750 or lower. To validate the sensitivity of the microarray, four different concentrations (0.1%, 0.03%, 0.01% and 0.004%) of HA peptide (YPYDVPDYA) were spotted. Individual arrays were incubated with murine monoclonal anti-HA antibody or normal murine IgG followed by secondary antibody (goat anti-murine IgG) conjugated to Cy3. Reactivity to HA peptide was dependent on anti-HA monoclonal antibody concentration and a dose dependent response against the antigen was observed with antibody concentration of 5 ng/ml or higher in each case except for the lowest concentration of HA which required >50 ng/ml of monoclonal antibody.

This example demonstrates the feasibility of this new technology in developing a peptide-small molecule microarray to assay for the reactivity of not only peptide specific autoantibodies but also reactivity against antibodies against a panel of haptens such as the xenobiotic compounds conjugated to peptide backbones. The peptide microarray technology disclosed herein may also be applied for fine epitope mapping. For example, the 4C8 is a monoclonal antibody that recognizes the inner lipoyl domain (128-229) of PDC-E2, but it did not react to any xenobiotics. Previous studies (see Migliaccio, C., et al. Hepatology 2001, 33:792), have shown the reactivity of the 2H4 clone requires both lipoic acid and the PDC-E2 inner lipoyl domain (128-229), whereas lipoic acid was not necessary for clone 4C8 or C355.1 binding. Although those three monoclonal antibodies showed disease specific apical staining pattern on bile duct (see Migliaccio, C. A. et al. J. Immunol. 1998, 161:5157; Migliaccio, C. A. et al. Hepatology 2001, 33:792), the clones 4C8, C355.1 and 2H4 recognize distinctly different epitopes within the PDC-E2 inner lipoyl domain. This peptide-small molecule microarray platform will be useful in defining the molecular requirement of chemical mimics involved in the breaking of tolerance in PBC and other indications.

The above approach for the preparation and use of chemical microarrays, has several advantages over previous devices and methods. Because the ligation reaction is highly site-specific and efficient, the mixing of a constant amount of ketone-scaffold with excess aminooxy-ligand generates large number of different ligand scaffold conjugates with identical levels of substitution. In addition, the ligand-scaffold conjugates, once prepared, can be stored and used for a long time. With these two unique features, high quality and normalized chemical microarrays may be generated that are comparable from sample to sample and from day to day. The microarray system is fully compatible with many biological assays. With a hydrophilic scaffold such as agarose, and a highly flexible hydrophilic linker, the ligands would be expected to be fully accessible to any cells, samples or analytes used in the analysis. The present invention enables one to easily print a mixture of ligands, with various ratios, into individual spots.

Although the foregoing invention has been described in some detail by way of illustration and example for purposes of clarity of understanding, it will be obvious that certain changes and modifications can be practiced within the scope of the appended claims. In addition, each reference provided herein is incorporated by reference in its entirety to the same extent as if each reference was individually incorporated by reference.

What is claimed is:

1. A microarray comprising a support having a plurality of discrete regions having a biopolymer spotted thereon, wherein attached to said biopolymer in each of said regions is a ligand that can be the same or different from a ligand in any other of said discrete regions, and wherein the concentration of said ligand in said discrete regions is substantially normalized.

2. The microarray of claim 1, wherein said support is selected from the group consisting of glass, polystyrene, PDVF membranes, nylon membranes, and polycarbonate slides.

3. The microarray of claim 1, wherein said biopolymer is a member selected from the group consisting of oligosaccharides, proteins, polyketides, peptoids, hydrogels, polylactates and polyurethanes.

4. The microarray of claim 1, wherein said biopolymer is attached to said support via noncovalent interactions.

5. The microarray of claim 4, wherein said noncovalent interactions are selected from the group consisting of hydrogen bonding, van der Waals interactions, hydrophobic interactions, hydrophilic interactions and combinations thereof.

6. The microarray of claim 1, wherein said biopolymer is attached to said support via covalent interactions.

7. The microarray of claim 1, wherein said ligand is selected from the group consisting of amino acids, peptides, proteins, sugars, lipids, nucleic acids, small organic compounds, pharmaceutical agents, candidate pharmaceutical agents, natural or synthetic antigens, and combinations thereof.

8. The microarray of claim 1, wherein said ligand is attached to said biopolymer via chemoselective ligation.

9. The microarray of claim 1, wherein said biopolymer is agarose, and said support is glass.

10. The microarray of claim 1, wherein said biopolymer is human serum albumin, and said support is polystyrene.

11. The microarray of claim 1, wherein the difference in concentration between any two discrete regions is less than 50%.

12. The microarray of claim 1, wherein the difference in concentration between any two discrete regions is less than 20%.

13. The microarray of claim 1, wherein the difference in concentration between any two discrete regions is less than 5%.

14. A method of producing a concentration-normalized ligand array, said method comprising:
(a) forming a ligand-modified biopolymer by attaching a ligand to a functionalized biopolymer via chemoselective ligation; and
(b) spotting an aliquot of said modified biopolymer mixture onto each of a plurality of discrete regions on a solid support to produce a concentration-normalized ligand array.

15. The method of claim 14, wherein said method further comprises, prior to step (b), the following step:
(a)(i) combining said ligand-modified biopolymer with a biopolymer solution to form a modified biopolymer mixture.

16. The method of claim 14, wherein said solid support is selected from the group consisting of glass, polystyrene, PDVF membranes, nylon membranes, and polycarbonate slides.

17. The method of claim 14, wherein said aliquot is spotted onto said solid support under conditions sufficient to form a gel-coated surface.

18. The method of claim 14, wherein said biopolymer is a member selected from the group consisting of oligosaccharides, proteins, polyketides, peptoids, hydrogels, polylactates and polyurethanes.

19. The method of claim 14, wherein said ligand is selected from the group consisting of amino acids, peptides, proteins, sugars, lipids, nucleic acids, small organic compounds, pharmaceutical agents, candidate pharmaceutical agents and combinations thereof.

20. The method of claim 14, wherein said ligand-modified biopolymer is peptide-modified agarose and said solid support is glass.

21. The method of claim 14, wherein said ligand-modified biopolymer is peptide-modified human serum albumin and said solid support is polystyrene.

22. A method for promoting cell or tissue growth at a desired site, said method comprising contacting said site with a ligand-modified biopolymer in an amount effective to promote cellular chemotaxis and cell or tissue growth at said site, wherein said biopolymer component is a member selected from the group consisting of agarose, polylysine and polyacrylamide, wherein said ligand component is a chemotactic peptide specific for a cell surface receptor, and wherein said ligand component is attached to said biopolymer component via chemoselective ligation.

23. The method of claim 22, wherein said biopolymer is agarose.

24. The method of claim 22, wherein said site is a member selected from the group consisting of a stent, a graft, an organ, a tissue and an implant.

25. The method of claim 22, wherein said cell or tissue growth occurs in vivo.

26. The method of claim 22, wherein said cell or tissue growth occurs in vitro.

27. A method for assaying the binding of ligands to a binding partner, said method comprising
(a) contacting a binding partner with a microarray of claim 1; and
(b) determining the amount of binding that occurs between said binding partner and the ligands present in the discrete regions of said microarray.

28. The method of claim 27, wherein said microarray comprises a modified agarose biopolymer.
